(19) United States
(12) Patent Application Publication
(10) Pub. No.: US 2025/0073402 A1
(43) Pub. Date: Mar. 6, 2025

(54) PLASMA AND GAS SYSTEM

(71) Applicant: CAPS Medical Ltd., Netanya (IL)

(72) Inventors: Boris KOGAN, Kiriat-Motzkin (IL); Ilan Oleg UCHITEL, Kfar-Saba (IL); Leonid YANOVITZ, Rishon LeZion (IL)

(73) Assignee: CAPS Medical Ltd., Netanya (IL)

(21) Appl. No.: 18/239,925

(22) Filed: Aug. 30, 2023

(51) Int. Cl. A61M 15/02; A61M 16/00; A61M 16/10; A61N 1/44

(52) U.S. Cl. CPC A61M 15/02; A61M 16/0003; A61M 16/022; A61M 16/10; A61N 1/44

(57) ABSTRACT

An endoscopically operated plasma delivery device, in which ionization gas also suffices to provide insufflation pressure allowing the device to be maneuvered in a treated body lumen. To assist in maintaining pressure safety while avoiding interfering with the parameters of plasma generation, ionization gas evacuation is automatically adjusted in response to monitored pressure in the body lumen. In some embodiments, rapid control response is assisted by augmenting ionization gas throughput with a second gas source, and using valved switching of the proportions of gas evacuated from the two sources. In some embodiments, a faster but cruder adjustment mechanism is coupled with a slower but finer-adjusting mechanism, potentially increasing responsiveness while maintaining control of equilibrium pressures. In some embodiments, direct manual adjustment of insufflation pressure is accommodated within the operational parameters of automatically adjusting control, optionally with override.

### FIELD AND BACKGROUND OF THE INVENTION

The present invention, in some embodiments thereof, relates to the field of plasma-based medical treatments and more particularly, but not exclusively, to cold plasma-based medical treatment.

Plasma is a general term encompassing compositions of ionized gas, generally including free electrons and ions, as well as neutral atoms and molecules, and often free radicals. Plasma may be produced by electric discharge through gas, causing gas atoms or molecules to be excited and ionize. Some applications of plasma are based on Dielectric Barrier Discharge (DBD) for generation of the non-thermal plasma of low temperature, or so-called “cold” plasma. Such cold plasma is a low-ionized and non-thermal plasma generated at atmospheric pressure conditions. It has been found that cold plasma can be used for various applications in medicine and industry.

### SUMMARY OF THE INVENTION

According to an aspect of some embodiments of the present disclosure, there is provided a system for controlling luminal pressure during plasma dosage to a luminal space within tissue, the system including: a plasma inputs generator, configured to provide to a plasma probe an ionization gas flow according to one or more parameters, the ionization gas flow sufficient to maintain the luminal space in an insufflated state; the plasma probe, sized for insertion to the luminal space, and configured to receive the ionization gas flow, along with an electrical signal an electrical signal delivering power for plasma generation, to generate plasma within the luminal space; at least one evacuation conduit, sized for insertion to the luminal space; a pressure transducer, configured to indicate gaseous pressure in the luminal space; and a gas control unit, coupled to the at least one evacuation conduit, and configured to maintain the indicated gaseous pressure in the luminal space within a targeted range of pressures by adjusting a rate of gas outflow from the luminal space through the at least one evacuation conduit, while plasma inputs generator continues to produce ionization gas flow according to the parameters.

According to some embodiments of the present disclosure, the gas control unit includes a pump and at least one valve; the pump operates to evacuate a gasses from the ionization gas flow mixed with gases from a second source bypassing the luminal space; and the gas control unit adjusts the rate of gas outflow from the luminal space by actuating the valve to adjust a relative amount of gasses from the ionization gas flow and from the second source.

According to some embodiments of the present disclosure, the gas control unit: operates to establish a low-pressure region with an exhaust pressure below atmospheric pressure, in non-equilibrium pressure communication with: the luminal space, and a second source of gas, bypassing the luminal space and at a pressure above the exhaust pressure; includes at least one valve regulating flow into the low-pressure region from at least one of the luminal space and the second source of gas; and adjusts the rate of gas outflow by operating the at least one valve.

According to some embodiments of the present disclosure, in a basal operating condition, flow from the second source of gas is at least 50% of the ionization gas flow passing into the low-pressure region with the exhaust pressure.

According to some embodiments of the present disclosure, the rate of exhaust of gasses through gas control unit from the low-pressure region with the exhaust pressure changes by less than 50% of corresponding changes in rate of gas flow gas outflow resulting from adjustment by gas control unit.

According to some embodiments of the present disclosure, optionally including any of the previous five groups of embodiments, the gas control unit maintains the targeted range of pressures by operation of a first pressure-control actuator and a second pressure-control actuator; wherein the first pressure-control actuator responds more rapidly than the second pressure-control actuator, and adjustment to the second pressure-control actuator has more controlled effects on pressure than adjustment of the first pressure-control actuator.

According to some embodiments of the present disclosure, at least one of the first and second pressure-control actuators is a valve, and at least one of the first and second pressure-control actuators is a pump.

According to some embodiments of the present disclosure, optionally including any of the previous seven groups of embodiments, a manual valve is provided, adjustable to modify gas outflow and change the gaseous pressure in the luminal space to a new pressure within the targeted range of pressures; wherein adjustment performed by the gas control unit corrects to allow the new pressure to remain.

According to some embodiments of the present disclosure, upon receiving an indication of a change from the new pressure toward a pressure beyond a threshold of the targeted range of pressures, the gas control unit adjusts the indicated gaseous pressure in the luminal space toward a pressure further from the threshold than the new pressure.

According to some embodiments of the present disclosure, a range of available adjustment of the manual valve is limited to prevent a manual change to a pressure outside the target range.

According to some embodiments of the present disclosure, optionally including any of the previous ten groups of embodiments, the at least one evacuation conduit includes at least first and second conduits; wherein inlets to the first and second conduits are separated along their axis of elongation so that inlets of the second conduit are positioned to collect fluid before the fluid reaches inlets of the first conduit; and where the first conduit carries the majority of exhaust flux of the ionization gas flow from the luminal space.

According to some embodiments of the present disclosure, optionally including any of the previous eleven groups of embodiments, gas control unit is configured to adjust the rate of gas outflow, based additionally on one or more sensed indications of the operation of at least one of an insufflator or respiratory machine.

According to some embodiments of the present disclosure, optionally including any of the previous twelve groups of embodiments, a conduit is provided, configured for delivery of material into the luminal space without going through the plasma probe and under control of the gas control unit; wherein the gas control unit adjusts the rate of gas flow to compensate for the delivered material.

According to an aspect of some embodiments of the present disclosure, there is provided a method of controlling luminal pressure during plasma dosage to a luminal space within tissue, the system including: inserting the plasma probe into the luminal space; operating a plasma inputs generator to provide to the plasma probe an ionization gas flow according to one or more parameters, the ionization gas flow sufficient to maintain the luminal space in an insufflated state, while the plasma probe generates plasma using the ionization gas flow and an electrical signal delivering power for plasma generation; and while the plasma inputs generator continues to produce ionization gas flow according to the parameters, evacuating the ionization gas from the luminal space through at least one evacuation conduit under the control of a gas control unit; wherein the evacuating includes repeatedly: receiving an indication of gaseous pressure in the luminal space; and adjusting a rate of the evacuating, based on the received indication, to maintain the indicated gaseous pressure in the luminal space within a targeted range of pressures, allowing the plasma inputs generator to continue providing the ionization gas flow according to the one or more parameters.

According to some embodiments of the present disclosure, the gas control unit includes a pump and at least one valve; and the evacuating includes evacuating a mix of gasses from the ionization gas flow and from a second source; and the adjusting includes actuating the valve to adjust a relative amount of gasses from the ionization gas flow and from the second source.

According to some embodiments of the present disclosure, the evacuating includes establishing a low-pressure region under the control of the gas control unit with an exhaust pressure below atmospheric pressure, and in non-equilibrium pressure communication with: the luminal space, and a second source of gas at a pressure above the exhaust pressure; and the adjusting includes adjusting least one valve regulating flow from at least one of the luminal space and the second source of gas into the low-pressure region.

According to some embodiments of the present disclosure, optionally including any of the previous three groups of embodiments, the adjusting includes actuating both a first pressure-control actuator and a second pressure control-actuator; wherein the first pressure-control actuator responds more rapidly than the second pressure-control actuator, and wherein adjusting the second pressure-control actuator has more controlled effects on pressure than adjusting of the first pressure-control actuator.

According to some embodiments of the present disclosure, actuating the first and second pressure-control actuators includes adjusting both a pump and a valve.

According to some embodiments of the present disclosure, optionally including any of the previous five groups of embodiments, the evacuating includes: receiving an indication that a valve has been manually adjusted to modify gas outflow and change the gaseous pressure in the luminal space to a new pressure within the targeted range of pressures; allowing the change in the gaseous pressure to remain as a new equilibrium pressure; receiving an indication of a further change from the new pressure toward a pressure beyond a threshold of the targeted range of pressures; and adjusting the indicated gaseous pressure in the luminal space toward a pressure further from the threshold than the new pressure.

According to some embodiments of the present disclosure, optionally including any of the previous six groups of embodiments, the at least one evacuation conduit includes at least first and second conduits; and including: positioning inlets to the first and second conduits with separation along their axis of elongation, so that inlets of the second conduit are positioned to collect fluid before the fluid reaches inlets of the first conduit.

According to some embodiments of the present disclosure, optionally including any of the previous seven groups of embodiments, the adjusting includes: receiving an indication of the operation of at least one of an insufflator or respiratory machine to modify the gaseous pressure; and adjusting the rate of gas outflow based also on the indication.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, controls. In addition, the materials, methods, and examples are illustrative only and are not intended to be necessarily limiting.

As will be appreciated by one skilled in the art, aspects of the present disclosure may be embodied as a system, method, or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object-oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer (e.g., as a stand-alone software package), partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer; and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the drawings:

FIG. 1 schematically represents a system for plasma delivery to a target treatment region with pressure, flow, and composition management, according to some embodiments of the present disclosure;

FIG. 2A schematically illustrates pressure states related to pressure regulation and safety, according to some embodiments of the present disclosure;

FIG. 2B schematically represents a system for plasma delivery to a target treatment region with manual (and optionally automatic) passive exhaust control using a tap, according to some embodiments of the present disclosure;

FIG. 3 schematically represents a system for plasma delivery to a target treatment region with active evacuation control, according to some embodiments of the present disclosure;

FIG. 4 schematically represents a system for plasma delivery to a target treatment region with bidirectional in-line gas control, according to some embodiments of the present disclosure;

FIG. 5 schematically represents a system for plasma delivery to a target treatment region with automated gas control in coordination with an external insufflator/respiratory machine, according to some embodiments of the present disclosure;

FIG. 6 schematically represents a system for plasma delivery to a target treatment region with automated evacuation control via inline vacuum, according to some embodiments of the present disclosure;

FIG. 7 schematically represents a system for plasma delivery to a target treatment region with soft start cross-valve, according to some embodiments of the present disclosure; and

FIG. 8 schematically represents a flowchart for plasma delivery to a target treatment region, according to some embodiments of the present disclosure.

### DESCRIPTION OF SPECIFIC EMBODIMENTS OF THE INVENTION

The present invention, in some embodiments thereof, relates to the field of plasma-based medical treatments and more particularly, but not exclusively, to cold plasma-based medical treatment.

### Overview

A broad aspect of some embodiments of the present disclosure relates to regulation of the internal gaseous environment of a target treatment region comprising an enclosed volume (a luminal space) including tissue to be treated with plasma.

In some embodiments and associated uses, the treatment comprises treatment by exposing an internal surface area of the enclosed volume to plasma. In some embodiments, the plasma used is low-temperature (“cold”) plasma, generated at temperatures compatible with physiological tolerance for the duration of exposure. This may be, for example, a temperature below a threshold of thermal injury (e.g., below 50° C.) and/or a threshold of partial protein denaturation. Duration of exposure may affect the limit of physiological tolerance. In some embodiments, the internal gaseous environment overall is maintained at a physiologically tolerable temperature; e.g., in a range of 30° C.-42° C. Ionization gas may begin at a temperature which is colder and warmed up during the process of plasma generation.

In some embodiments, regulation of pressure is of particular concern, e.g., to avoid damage to the treatment target region itself, and/or other regions to which pressurization may escape. Optionally, one or more additional aspects of the gaseous environment such as gas temperature and gas composition (e.g., presence of plasma-modified species such as water, nitrogen and/or oxygen) are regulated; for example, to ensure safe operation and/or to augment generation of secondary reactive species produced by the plasma.

In some embodiments, the enclosed volume comprises the lumen of a bladder.

In some embodiments, the enclosed volume comprises a luminal region of the gastrointestinal tract; for example, a stomach, colon, or rectal cavity.

In some embodiments, the enclosed volume comprises one or more lungs, regions of a lung, and/or airways of a respiratory system.

Other examples of organs and/or regions comprising the enclosed volume include: a bile duct, a kidney, a peritoneal space, a sub-cranial space, a nasopharyngeal space (e.g., sinus), a larynx, an esophagus, a uterus, and a maxillofacial space.

These spaces are unified in that they are accessed or potentially accessed through lumens and/or apertures of sizes which impose restrictions on gas flow, to the extent that gas flow potentially over-pressurizes them without due care in its management. Different such regions may differ particularly in their volume; potentially also in their sensitivity to pressure (e.g., the acceptable range of operating pressures used with them).

Typically, for consistent plasma generation and delivery, a high discharge voltage is provided to the site of plasma generation, along with a flow rate of ionization gas (the plasma gas medium) maintained within a relatively narrow range. Ionizing gas flow rate affects properties of generated plasma. Additionally or alternatively, embodiments of the present disclosure depend on ionizing gas flow rate as a component of thermal regulation (e.g., by being matched to the level of energy input, and/or to carry heat away from the probe and/or target treatment region).

In some embodiments, the ionization gas flow is predetermined; that is, generated according to predetermined operating parameter(s) determined according to plasma treatment requirements. In particular, variable control of the predetermined ionization gas flow in response to pressure changes inside the luminal space of the target treatment region is preferably avoided, except in exception circumstances outside of normal operation (e.g., a system safety shutdown, a system safety switch to a recovery mode, or a switch to a standby mode). The predetermined operating parameter(s) of ionization gas flow optionally comprise one or more time-dependent parameters; for example, parameter(s) defining the ionization gas flow as constant, pulsed, and/or cycling. The predetermined ionization gas flow is predetermined in magnitude (directly and/or indirectly); for example, predetermined in terms of operating pressure, sensed pressure, duty cycle, pump voltage, valve inlet state, and/or sensed rate of flow.

An aspect of some embodiments of the present disclosure relates to maintaining fine control of pressure in an ionization gas-insufflated target treatment region, without disturbing delivery of inputs determining characteristics of plasma being generated using the ionization gas. The ionization gas, in some embodiments, comprises one or more ionizable species (e.g., helium, neon, air, CO₂ and/or argon), in sufficient concentration and suitable admixture with other species so as to promote ionization to plasma in the presence of an energetic source such as a high voltage (e.g., about 500 V or higher) frequency-varying (e.g., at a frequency of 100 KHz or higher) electrical field. The high potentials and slew rates this produces result in ionization in the ionization gas, including associated transfer of energy which produces heating. Secondary species may be formed (e.g., by nitrogen and/or oxygen) upon interacting with ions created in the plasma; these species potentially contribute to therapeutic effects of plasma.

In some embodiments of the present disclosure, a plasma generating device includes a control mechanism that maintains a targeted gas pressure or range of gas pressures inside the target region, without altering ionization gas and plasma flow parameters, and using the ionization gas itself as a primary or sole source of insufflation pressures, sufficient to enable, for example, visualization and/or probe maneuverability inside the target region.

The flow rate of ionization gas, in some embodiments, is rather high relative to the enclosed volume; e.g., up to several replacements per minute (further examples given below). For example, in some embodiments, the enclosed volume is the lumen of a bladder, with an internal (and optionally insufflated) volume of about 200-600 ml. Many of the above-mentioned luminal spaces will have a smaller internal volume; some may be larger.

In bladder, the working pressure, also referred to herein as gauge pressure (i.e., pressure above atmospheric pressure, which corresponds to about 760 mmHg at sea level) is typically in an operating range of about of 5-20 mmHg. It may preferably be kept in an operating range nearer to a lower portion of that range, e.g., 5-15 mmHg, 5-12 mmHg, or 7-12 mmHg. Optionally, there is targeted baseline value and/or targeted baseline range within (e.g., offset from either end of and/or near the middle of) the operating range; for example, in the range of about 8-10 mmHg.

In some embodiments, an appropriate flow rate of ionization gas is, for example in a range of 0.1-10 l/min; e.g., about 0.5, 1, 2, 4, 6, 8 or 10 l/min. Accordingly, an empty bladder (for example) may be filled to its working pressure in, e.g., hundreds of milliseconds to a few seconds (e.g., as quickly as 500 msec or less, unless limited during startup) upon activation of a plasma probe within it. A matching exhaust of gas helps ensure that safe working conditions are maintained. In some embodiments, exhaust of non-gaseous material is provided; for example: exhaust of fluids and/or solids introduced and/or present in the treatment target region. Optionally, there are arrangements (e.g., special positioning of evacuation inlets) to divert this evacuation component to a secondary channel of evacuation, to mitigate disturbance to the equilibrium of the flux of the ionization gas flow.

Inside the body, however, access to the target region is generally limited to vents occupied by and/or and made available using minimally invasive tools such as endoscopes, trocars, and/or needles. Accordingly, the trapped gas needs to be vented continuously through at least one of these devices, optionally in a controlled manner and/or vented against constant pressure through an access port. Other substances also may need to be evacuated; preferably without disturbing control of the flux of ionization gas flow.

Critically, the working margin of insufflation variation is typically much smaller than the targeted working volume; e.g., less than 2%, 1%, or 0.5% of the total volume in at least one direction (over-pressure or under-pressure). This potentially corresponds to a change of about 0.5-2 ml of gas at standard pressure when the working volume is about 200 ml (and, moreover, substantially non-compliant to pressure changes over short periods and/or within the targeted pressure range). In some embodiments, the ratio of gas flux per minute to volume in the enclosed volume of the target treatment area itself is at least one. In some embodiments, a ratio of gas flux per minute to volume in the enclosed volume of the target treatment area itself is between about 0.5 and 30; for example, a ratio of 5, 10, 15, 20 or more. Optionally it is higher.

It may be understood, especially under such circumstances, that several types of variations in conditions may result in relatively rapid changes in the balance of gas inflow and outflow. For example, as instruments are moved, the quality of the sealing of tissue around them may suddenly change, resulting in uncontrolled leakage and/or its cessation/reduction. The presence of loose materials inside a body cavity (e.g., the target treatment region itself) may produce obstructions. For example, the mass and/or viscosity of a drop of contaminating fluid may alter the evacuation rate of gasses; transiently (e.g., for just a few milliseconds up to a few seconds), or permanently (e.g., requiring plasma delivery to shut down and/or enter a recovery mode). Obstructions potentially comprise solid material disturbed from the target treatment region or otherwise introduced. Mechanical variability in device function (e.g., in response to a brownout) potentially occurs. Potentially, a conduit's movement (e.g., kinking) affects its resistance and/or pressurization. Potentially, a hollow organ itself moves, e.g., by contracting or expanding, or is affected by the movement of another internal organ. Potentially, external pressure is placed on the volume of the target treatment region, e.g., to judge a level of insufflation, or for another reason.

Accordingly, in some embodiments, and considering in particular potential worst cases, there are potentially only a few milliseconds of time available (e.g., 5-200 msec; for example, 50 msec) to detect and react to a developing over-pressure (dangerous to the organ) or under-pressure (resulting in lumen collapse sufficient to interfere with probe positioning and/or target visualization).

Somewhat relieving constraints, a rate of change in pressure (e.g., a rate of change of inflow relative to outflow) is likely to take somewhat longer to fully develop; for example, with a rapidity of several tens to several hundreds of milliseconds (e.g., 50-500 msec), or a longer period, which potentially provides more time to begin and establish control responses.

In some embodiments (e.g., plasma treatments in lung), ionization gas supplied through the plasma probe is mixed with shifting volumes and/or pressures of gas determined by another device, e.g., a ventilator. This potentially complicates the issue of maintaining a suitable pressure inside the target region.

While ionization gas flow rate can in principle be stopped (although stopping itself may be a matter of a short but significant time; for example, 50-100 msec), reduction of ionization gas flow rate while maintaining targeted conditions of plasma production is potentially unavailable for a variety of reasons.

For example, this flow rate, in some embodiments, is determined by operating parameters at a level expected to ensure plasma remains at a safe temperature (e.g., within one or more of the non-thermal regime criteria mentioned above).

In another example, discharge characteristics of the plasma device are potentially altered (e.g., adversely) if the concentration of plasma is not maintained at a predetermined level by the flow of gas. In another example, characteristics of plasma that reaches the target are potentially altered (e.g., adversely). For example, plasma characteristics can be sharply time-dependent (e.g., changing significantly in concentration and/or composition over the course of a few microseconds or milliseconds), and so also affected by the rate of ionization gas which carries it out of the plasma generating probe.

Furthermore, even stopping plasma generation is potentially difficult and/or risky to perform instantaneously; limited, e.g., by constraints on the dissipation of delivery lumen gas pressure, probe and/or plasma temperature, and/or electrical potential. In some embodiments, electrical shutdown must practically precede ionization gas flow shutdown (e.g., to avoid overheating), potentially adding time to the shutdown process.

Accordingly, in some embodiments of the present disclosure, much of the responsibility for maintaining an appropriate insufflation pressure in a body lumen is shifted to the control of gas evacuation, particularly for specifically low-latency control over insufflation pressure.

For example, low-latency control is provided to exert compensation to overcome a detected change in intraluminal pressure within a period of 100 msec or less, 50 msec or less, 20 msec or less, 10 msec or less, 5 msec or less, or another brief period. “Overcome” means that a movement toward a threshold limit is wholly stopped, and potentially reverse.

In another example, additionally or alternatively, low-latency control may physically begin compensation to overcome a detected change in intraluminal pressure within a period of, for example: 100 msec or less, 50 msec or less, 20 msec or less, 10 msec or less, or 5 msec or less. “Begin” for this purpose means that there is sufficient actuation of an element such as a valve that an effect on the pressure trend may be noticed in ongoing pressure measurements. The time is potentially determined by mechanical limitations; e.g., a digital control loop may be capable of reacting within 1 msec of a determined need for adjustment, but a pressure-control actuator such as a valve or pump may actuated over a considerably longer period of time. Lags in the pressurized system itself potentially also play a role in time delays, for example, a delay while a certain volume is sufficiently de- or re-pressurized so as to produce the targeted response.

The response time for evacuation is selected, in some embodiments, so that pressure enters never reaches a level from which a sudden 100% blockage of flow would result in an over-pressure situation (e.g., above 12 mmHg, 14 mmHg. 16 mmHg, 18 mmHg, or 20 mmHg) before plasma generation can be safely shut down, including shutdown of ionization gas delivery. Assuming, e.g., 1-5 msec for detection and 50-100 msec for total shutdown, this means maintaining at least a 55-105 msec safe-shutdown margin, judged, e.g., against a worst considered case characterized in terms of magnitude and/or suddenness. The margin is optionally increased as is found suitable, e.g., simply to increase a safety factor, according to regulatory requirements, and/or further failure mode considerations. Examples of such considerations include a scenario combining external pressure on the organ coupled with blockage of gas evacuation, a scenario in which sensing from a fallback sensor is relied upon to initiate shutdown, a scenario in which sensor polling and/or interrupt response is delayed (e.g., by a coincidental event), and a scenario in which ionization gas flow inadvertently increases.

Herein “passive” gas evacuation refers to evacuation driven by a difference from ambient (e.g., room air) pressure. “Active” gas evacuation refers to evacuation in which a lower-than-ambient pressure is established to develop a larger pressure gradient (speeding up evacuation). “Manual” and “automatic” control of gas evacuation potentially relate to either passive or active gas evacuation. “Manual” control refers to gas evacuation adjustment by operator inputs, which may be mechanical (e.g., physically manipulating a valve), or signal based (e.g., electronic signal based in response to manipulating a controller). “Automatic” control refers to changes which are introduced by at least partially autonomous operation of a machine according to its present parameters and optionally sensing data. The parameters of automatic control are optionally set themselves manually, automatically, and/or according to predetermined values. Embodiments may be entirely manual or automatic, but it is also envisioned that embodiments of the present disclosure may provide mixed manual and automatic controls over the homeostasis of the gaseous environment of a target treatment region, e.g., different types of control for two or more of pressure, temperature, and/or composition, and/or each of the two type of control for at least one of these parameters of the gaseous environment, or another parameter of the gaseous environment.

As a matter of system design, consideration of a rapid response requirement has lead the inventors to consider additional potential issues. For active gas evacuation, for example: pumps suitable for medical use such as membrane (diaphragm) pumps or peristaltic pumps are potentially unsuitable for rapid startup/shutdown, or even rapid increases/decreases in pumping volume. Such pumps may operate less consistently (e.g., with more pulsations) under inconstant load. In some embodiments of the present disclosure, operation under constant load is provided by suitably balancing ionization gas evacuation from the target treatment region with the evacuation of ambient air or another source of gas (“bypass” gas). This comprises, for example, an arrangement in which one or more valves operate to change the rate at which gas is evacuated from a target treatment region, without significant change in the load experienced at the pump itself. Accordingly, the valve state becomes a controller of evacuation rate, optionally in addition the pump itself. Preferably, the valve may be placed to minimize the “dead volume” in evacuation conduits outside of the target treatment region, potentially further minimizing control lag.

Opening a valve (increasing the coupling of the enclosed volume to a lower pressure environment) may result in a rapid response onset. For example, valve operation itself takes about 10-500 msec; optionally about 20-50 msec, 20-100 msec, 50-100 msec, 50-200 msec, or 100-200 msec; optionally it completes within 20 msec, 50 msec, or 100 msec.

Optionally, the new rate of steady-state (equilibrium) flow achieved with active gas evacuation regulation is optionally regulated by additionally modifying (albeit with potentially greater lag) the operation of a pump or other pressure controlling device itself. For example, a rate of pressure decline in the target treatment area may be measured following valve adjustment, and the rate of pumping (rate of operation of the pump itself) adjusted accordingly. Optionally, an early detection of a trend in pressure is extrapolated using assumptions conservative of safety, and reacted to accordingly with aggressive initial mitigating adjustments. If the trend proves less extreme (e.g., less dangerous) over time, mitigating adjustments are optionally reduced and/or reversed accordingly.

Optionally, pump operation is fine-tuned as appropriate based on sensing, as appropriate to the accuracy and precision of its known operating parameters. In some situations and/or for some evacuation elements, the relationship of operating state to environment state and effect on evacuation rate may be relatively unknown (e.g., difficult to calibrate, unstable, or otherwise unavailable). Furthermore, there may not be sufficient time in a rapid-response regime to determine this based on feedback measurements. For example, it may not be known with certainty what the trend of a sudden imbalance in inflow/outflow rates is; e.g., whether it is transient, about to increase rapidly, or staying the same.

Accordingly (e.g., continuing the pump/valve example), it may be easier to reach an inflow-matching evacuation rate (e.g., faster and/or with fewer control oscillations) by modifying pump operation than by modifying valve operation, even if the valve itself has a faster initial response time.

It is noted that mixing with ambient gas may make determination of actual evacuation rate difficult even for a pump, unless additional measurements are performed, e.g._, to determine ionization gas concentration (spectrographically, for example), present working temperature, and/or to determine a rate of ambient (or other auxiliary) gas delivery. The latter is potentially easier than for the case of determining ionization gas flow, since the equipment potentially need not be placed in the body-contaminated pathway, and/or potentially need not be as space-constrained.

While a variable and precisely controllable (i.e., continuously variable) valve is optionally provided in some embodiments, it may be difficult to determine or predict results to the requisite degree of precision that re-establishes or maintains a given level of pressure in the target treatment region. For example, when operating with rapid loop times (e.g., 5-100 msec) it may be difficult to eliminate oscillation due to cybernetic (feedback) lag. It is also noted that a precisely operating valve may not be a fast operating valve. For example, a valve given precision by operating over several turns of a screw may require a longer period of actuation in order to open/close to some requisite degree. A ball valve actuating over 90° of arc, in contrast, is potentially faster to operate, but may be difficult to position accurately.

Additionally or alternatively, it is a potential advantage to cost and/or reliability to distinguish between rapid-onset pressure adjustment functionality provided by a first element (e.g., valve operation with a crudely selectable state and/or poorly characterized effect on evacuation rate) and slower but potentially more precise pressure adjustment functionality provided by a second element (e.g., an electrically controlled pump and/or precision valve). For example, a cheap, disposable valve may be preferable to an expensive valve in need of frequent reprocessing (e.g., sterilization). Conversely, in some embodiments, a pump with cruder controllability is optionally used to augment the control characteristics (e.g., dynamic range) of valve-regulated fine control.

In accordance with the foregoing: in some embodiments, adjustment of a rate of evacuation from a target treatment region comprises adjusting a first element to rapidly modify the rate of evacuation in a known direction, together with adjusting a second element to correct for the modification performed by the first element.

In another example, one or more valves may be provided with an on-off operation; opening to provide a potentially low-latency and step-wise increase in evacuation rate, which is then adjusted to a needed rate by modifying a slower adjustment (e.g., rate of pump operation). Restoration of the valve (and its “rapid response” capability) to a closed state may be achieved, e.g., by decreasing a duty cycle of its open state while suitably modifying pump speed to match. This has the potential disadvantage of introducing a period of “clatter” as the valve changes between states after triggering. This may still comprise a relative potential advantage over constant adjustment of an intermediate-value valve duty cycle. In other words, a relatively slow-adjusting element may be used to set the equilibrium evacuation rate, such that a relative rapid-adjusting element can be kept usually inactive, but prepared for activation in case a risk threshold for over-pressure is crossed.

In some embodiments, a plurality of valves is provided, and optionally opened/closed in appropriate groupings and/or duty cycle arrangements to achieve a targeted evacuation rate (again, subsequent pump-regulated or other slower compensation may be introduced). Optionally, each of the plurality of valves regulates a different respective gas evacuation capacity change, e.g., differing by powers of two to allow stepwise selection of a plurality of evacuation rates. Optionally, both a finer-adjusting and cruder-adjusting valve is provided.

In some embodiments, additionally or alternatively to the above measures, a low-pressure tank is provided as a buffer between (and separating) the pump and the target treatment region as a way of ensuring an “instantly available” suction boost. The pump may be used to keep the low-pressure tank at its operating pressure. However, such a tank has its own pressure “inertia”, which may slow and/or complicate control compared to what is available with an electrically operated pump. Conversely to being used as a rapid boost, such a tank is optionally used as a source of bias pressure to modify a set point of pressure used to control a rate of evacuation. Optionally, such a source is provided in place of a pump.

In some embodiments (again, additionally or alternatively to the above measures), a low-pressure tank or other low pressure source (e.g., building facility vacuum port) is provided in parallel to a directly pumped-out evacuation line leading from the target treatment area, and valved open as appropriate to provide rapid boosting of momentary evacuation capacity. This has the potential advantage of preserving a line allowing the pump to directly adjust the rate of exhaust, while also providing an option for a rapid (e.g., valve-controlled) boost to rate of evacuation/exhaust. In some embodiments, valving of pressure from a similarly configured over-pressure tank (or other higher pressure source) may serve a role in exhaust rate control (additionally or alternatively). For example, this tank could be used to quickly reduce the rate of evacuation in case pressure measurements indicate that the volume of the target treatment region is at or nearing insufflation collapse.

In some embodiments, the same concept of low-latency pressure control coupled to longer-latency precision of control is used to control steady state or slowly-varying levels of ionization gas passive evacuation from the target treatment region. For example, there may be a “slow” valve and a “fast” valve. Additionally or alternatively, a portion of gas evacuation may be manually controlled. The rate of evacuation may be controlled, e.g., by a setting of a valve and/or stopcock. This can be used to manually adjust the system's equilibrium point (e.g., level of insufflation) optionally with automatic control superimposed using a more rapidly adjusting device.

Before explaining at least one embodiment of the present disclosure in detail, it is to be understood that the present disclosure is not necessarily limited in its application to the details of construction and the arrangement of the components and/or methods set forth in the following description and/or illustrated in the drawings and/or given in the Examples. Features described in the current disclosure, including features of the invention, are capable of other embodiments or of being practiced or carried out in various ways.

Plasma System with Plasma Generator and Gas Control Unit

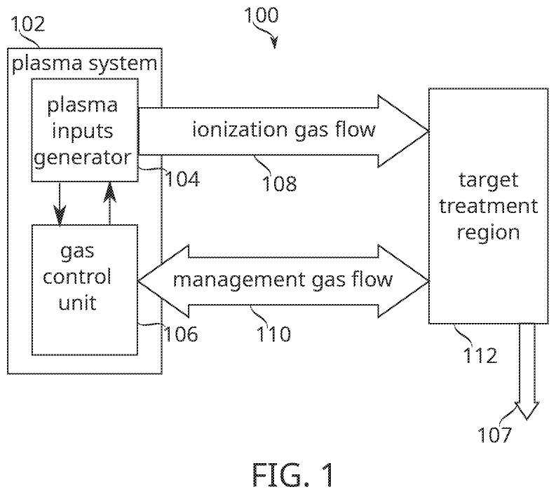

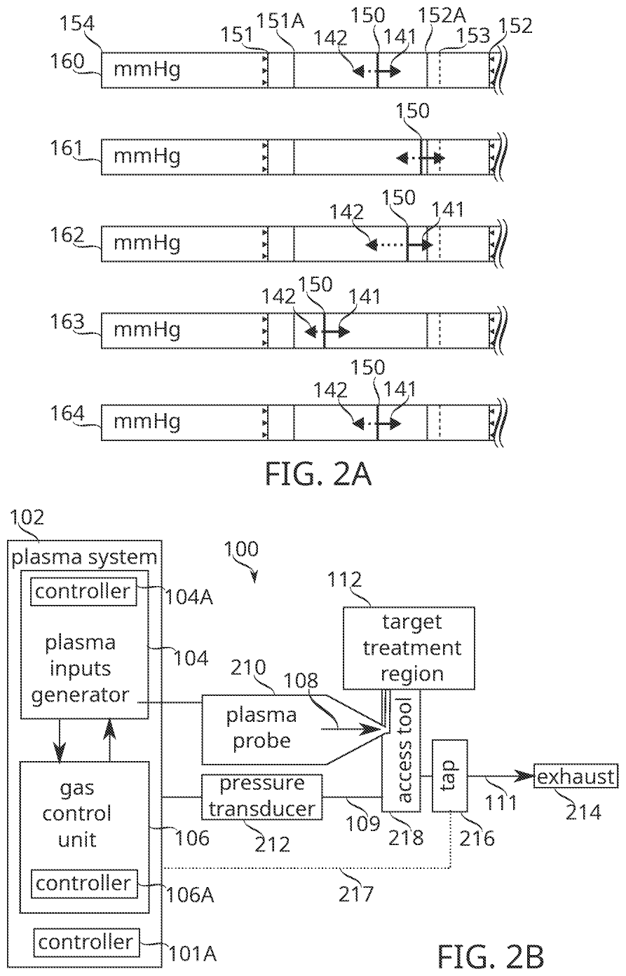

Reference is now made to FIG. 1, which schematically represents a system 100 for plasma delivery to a target treatment region 112 with pressure, flow, and composition management, according to some embodiments of the present disclosure. In some embodiments, system 100 is configured for use to deliver plasma internal to a living body, e.g., to a lumen of an organ and/or an insufflated space such as a peritoneal space. In some embodiments, system 100 more specifically delivers plasma under endoscopic observation. Preferably, plasma delivery is performed under minimally invasive conditions, e.g., using natural body orifice(s), and/or surgical orifices only just large enough for insertion of an endoscopic device. For example, the plasma delivery conduit itself (e.g., a part of plasma probe 210, as also discussed in relation to FIG. 2B) may have an outer diameter of about 1.5 mm, 2 mm, 3 mm, 4 mm, 5 mm, 8 mm, or 10 mm or less (e.g., diameter of about 1.5 mm-2.0 mm). In some embodiments, gas evacuation is performed (simultaneous with plasma delivery) through a conduit having an outer diameter less than 10 mm; optionally no larger than the outer diameter of the plasma delivery conduit, or no larger within a factor of 2×. Length of the plasma delivery conduit is sufficient to reach a target organ through its selected natural and/or surgical orifice; e.g., to reach a bladder through urethra. For example, in some embodiments, the plasma delivery conduit is at least 30 cm, 40 cm, 50 cm, 60 cm, 80 cm, 100 cm, 120 cm, 150 cm, 180 cm, or 200 cm long.

In some embodiments, target treatment region 112 comprises an enclosed volume including tissue to be treated with plasma (e.g., treated by exposing an internal surface area of the enclosed volume to plasma). Due to the enclosure, gas supplied to target treatment region 112 is retained, except as released in the management gas flow 110 and/or by optional and/or unintended leakage 107.

In the schematic of FIG. 1, plasma system 102 includes plasma inputs generator 104 and gas control unit 106. Plasma inputs generator 104 provides, in particular, a controlled ionization gas flow 108 (that is, the gas medium in which plasma delivered to target treatment region 112 is ignited), as well as performing other functions such as generating suitable electrical signals for plasma generation. Plasma itself is preferably created within a plasma probe (e.g., plasma probe 210 as depicted in FIGS. 2B-5), at a location where ionization gas and the electrical signals for plasma generation (the two primary inputs needed for plasma generation) are exposed to each other. This location is generally near a distal gas discharge aperture of the plasma probe which is within or close proximity to target treatment region 112, so that the travel distance between site of generation and target is minimized sufficiently to overcome plasma decay. In FIG. 1, the plasma probe through which ionization gas flow 108 enters to target treatment region 112 is not shown, to emphasize flow control aspects of the embodiment shown. Plasma inputs generator 104, in some embodiments, comprises computer circuitry and/or programming instructions (e.g., of a programmable controller 104A and associated digital memory which is part of the computer circuitry) to measure, monitor and/or control its functions. Optionally, plasma inputs generator 104 is configured through a user interface which may comprise a graphical user interface of a computer, and/or appropriate mechanically operated controls (e.g., knobs, sliders, and/or buttons).

Plasma system 102 also includes gas control unit 106, which provides one- or two-way control of the management gas flow 110 into target treatment region 112. For example gas beyond that supplied in the ionization gas flow 108 is optionally added to support patient respiration, when target treatment region 112 comprises a portion of a lung (e.g., at a targeted and optionally varying pressure and/or volume). Additionally or alternatively, gas is removed to maintain that targeted pressure/volume and/or another parameter of the gaseous environment, and particularly as compensation for ionization gas flow 108. As examples of other parameters: gas is optionally added and/or removed via management gas flow 110 to help maintain an environmental condition such as temperature, humidity, liquid levels (e.g., acrosoled liquids), concentration of one or more species within the ionization gas, and/or concentration of plasma-reactive species in the environment such as oxygen and/or nitrogen, which are potentially involved in the production of reactive oxygen/nitrogen species (RANS).

Optionally, the functions of gas control unit 106 include sensing functionality, e.g., pressure, temperature, and/or composition sensing.

Gas control unit 106, in some embodiments, comprises computer circuitry and/or programming instructions (e.g., of a programmable controller 106A and associated digital memory which is part of the computer circuitry) to measure, monitor and/or control its functions. Optionally, gas control unit 106 is configured through a user interface which may comprise a graphical user interface of a computer, and/or appropriate mechanically operated controls (e.g., knobs, sliders, and/or buttons).

Optionally, controllers 104A, 106A are in further communication with a central controller 101A. Optionally, the functions of controllers 104A, 106A are provided as functions of central controller 101A.

It should be understood that physical packaging of gas control unit 106 and plasma inputs generator 104 is optionally in any suitable configuration, e.g., separately enclosed, or together in a single enclosure of plasma system 102. Some elements contributing to the functions of these elements may be packaged separately: e.g., elements such as sensors, user controls and/or control circuitry; and/or a plasma probe and/or gas supply tubing leading to/from target treatment region 112). For example, the exhaust functionality of gas control unit 106 may be distributed among a plurality of separately packaged outlets, e.g., a passive exhaust and an actively pumped outlet.

Uni- or bi-directional management gas flow 110 via gas control unit 106 is optionally is passively and/or actively controlled (automatically) with appropriate sensors (e.g., pressure sensor(s), temperature sensor(s), and/or chemical environment sensors(s)). Optionally, manual control is assisted by making this sensed data available for an operator (e.g. using a computerized user interface, not shown).

In some embodiments, gas control unit 106 communicates by exchange of signals with plasma inputs generator 104 (e.g., signals electrically, electromagnetically, or otherwise transmitted) as part of maintaining environmental control (e.g., pressure control) over the target treatment region. For example, monitoring of plasma real-time performance metrics potentially results in automatic and/or manually determined decisions to increase or decrease the flow of ionization gas flow 108 from plasma inputs generator 104, and/or to change the environment in the target treatment region. Such parameters are preferably not varied reactively, in some embodiments of the present disclosure (e.g., selected and/or predetermined), but even in this case, there is a potential under certain circumstances that device operation is changed reactively in order to achieve those parameters; for example, to maintain a parameter which corresponds to a sensed temperature, pressure, and/or flow rate detected on a distal side of a plasma probe and/or elsewhere in the system.

Control communication allows gas control unit 106 to change its operation accordingly to maintain targeted environmental conditions (e.g., gas pressure, liquid amounts, and/or gas/liquid composition). Optionally, gas control unit 106 exchanges signals with plasma inputs generator 104 to modify operation affecting plasma delivery, e.g., according to sensing data, and/or available limits on the control of management gas flow 110. For example, a shutdown signal may be exchanged should it be determined that a safety limit is exceeded. In some embodiments, leakage 107 is sensed (directly or indirectly), and included in calculations which estimate environmental conditions and/or determine modifications to performance which will maintain/bring about targeted environmental conditions.

In some embodiments, gas control unit 106 is provided with means for producing forced inflow, forced outflow, and/or forced plasma flow re-routing from the target treatment region. Optionally, gas control unit 106 controls, exchanges sensing/status signals with, and/or is configured to take into account an external gas source such as insufflator device or a respirator which may also be operating to manage a target treatment region; for example, insufflator/respiratory machine 502, further discussed in relation to FIG. 5.

An insufflator/respirator machine 502 and gas control unit 106 optionally operate together either independently or under coordinated control. For example, gas control unit 106 may provide electrical control signals to the insufflator device or respirator, or otherwise adjust its operation. In some embodiments, gas control unit 106 is configured to optionally shunt (and further optionally to reverse) ionization gas flow 108 itself.

As an example of a use for such a capability: a required period for probe shutdown may be extended by a requirement to maintain at least some flow through a plasma probe. Ionization gas flow 108 is optionally modified for some portion of that period via a junction between management gas flow 110 and ionization gas flow 108.

### Pressure Regulation Scenarios

Reference is now made to FIG. 2A, which schematically illustrates pressure states related to pressure regulation and safety, according to some embodiments of the present disclosure.

### Conventions, Implementation Options

FIG. 2A shows a series of pressure states 160 and 161-164, each of which indicates the settings of various pressures and limits which, in some embodiments, relate to pressure safety within a target treatment region 112. Elements of state 160 are fully labeled for purposes of explanation. For clarity, pressure states 161-164 are unlabeled for many of their elements, but corresponding elements remain the same.

The left side 154 of each bar represents 0 mmHg pressure difference with ambient (e.g., room) pressure. In passive evacuation systems, this is also the pressure against which evacuation works. In active evacuation systems, a lower pressure may be available, potentially increasing the maximum rate of ionization gas evacuation (i.e., the outgoing component of management gas flow 110, optionally realized as gas exhaust flow 111 (e.g., FIG. 2B)

Current pressure level 150 represents a pressure at a certain moment (e.g., a measured pressure inside and/or correlated with pressure inside target treatment region 112), subjected to moment-to-moment change according to the relative balance of gas inflow 141 (tending to increase pressure), and gas outflow 142 (tending to reduce pressure). The relative sizes of the arrows for gas inflow 141 and gas outflow 142 indicate their balance, and may also be taken as indicative of how much the pressure level 150 would change in a given period of time (e.g., 5 msec, or another period), were the opposing direction of flow not present.

Pressure limit 151 is a minimum acceptable pressure level, corresponding, e.g., to a limit below which there is insufficient confidence that a target treatment region 112 is adequately insufflated to allow proper probe maneuvering and/or observation, and/or maintain treated area/organ functionality. Optionally, this is treated by the device as a shutdown pressure limit should it be reached inadvertently during device operation; e.g., a pressure indicative that there is the potential for a serious fault in sensing and/or device positioning, and/or actually a fault in pressure regulation.

Pressure limit 152 represents a maximum acceptable pressure level, corresponding, e.g., to a limit above which there is judged to be risk of damage to the target treatment region 112 due to overpressure. It should be understood that pressure limit 152 may be set to leave a further margin of safety above it. For purposes of device function, however, pressure limit 152 is taken to be “absolute” in the sense that device operation makes every available effort to ensure that pressure limit 152 is not exceeded. However, although treated as absolute in the sense of safety, it may not actually be known absolutely; e.g., it may be characterized as being at least beyond a maximum pressure which the pressure management system allows when operating as intended. An example of this sort of definition is explained further in relation to failure mode pressure limit 153, hereinbelow.

Also shown are pressure limits 151A, 152A, which respectively correspond to minimum and maximum values of a target range of operating pressures within target treatment region 112. Optionally, these are set the same as respective pressure limits 151, 152 (and so also operational cutoff pressures); but conceptually they are potentially offset to lie within the cutoff range pressure limits 151, 152 define. In some embodiments, pressure limits 151A, 152A serve as indicators of how aggressively pressure regulation is pursued; e.g., more aggressively as current pressure level 150 approaches them, and/or passing outside of the range they define. In some embodiments, target operating pressure is defined otherwise; for example, as a single pressure which automatic pressure regulation continuously seeks to restore. A potential advantage of allowing a range of pressures is that manual adjustments (e.g., to valve settings) intended to adjust insufflation pressure can be applied without necessarily triggering an immediate automatic response to restore a pressure set point.

Nevertheless, in some embodiments and/or operating modes, there is additionally a pressure set point pressure (not shown) which automatic pressure regulation seeks to maintain. For example, manual adjustments resulting in a change to current pressure level 150 may be detected, and device operation self-adjusted to accept the adjusted position of current pressure level 150 as a new pressure set point (i.e., so long as it remains within pressure limits 151A, 152A and/or pressure limits 151, 152, according to the embodiment).

For purposes of explanation, pressure limits 151, 151A, 152A and 152 are treated as fixed in the examples of FIG. 2A; but they are optionally adjustable parameters of device operation, e.g., defined as offsets above and below a current pressure set point, and adjusted according as the pressure set point is adjusted. Additionally or alternatively, pressure limit 152A is optionally adjusted (e.g., lowered) as necessary to avoid encompassing a failure mode pressure limit 153, described hereinbelow.

For simplicity, in the further examples of FIG. 2A, the potential presence of a specific pressure set point is not discussed, as the examples relate to the handling of non-equilibrium conditions challenging the limits of pressure regulation.

Pressure limit 153 represents an optional “failure mode” pressure, optionally one of a plurality. This pressure, though not necessarily inherently unsafe, represents a pressure level beyond which the prevention of passing beyond pressure limit 152 is not guaranteed in the case of one or more device faults occurring (e.g., suddenly occurring). Accordingly, in some embodiments, the event of reaching pressure limit 153 results in the initiation of plasma delivery shutdown, in order to assure that no further event could cause pressure limit 152 to be exceeded.

The relationship between pressure limit 153 and pressure limit 152 is not necessarily known absolutely; e.g., pressure limit 153 may be set conservatively low so that the maximum acceptable pressure limit 152, whatever it is, is at least never (reasonably) exceeded, given limitations on how quickly the plasma delivery device can be shut down, and reasonable assumptions about worst case conditions (further discussed below).

Pressure limit 153, in some embodiments, is “floating”, e.g., adjustable and optionally automatically recalculated according to present conditions. For example, when inflow 141 is slower (shorter arrow), pressure limit 153 is optionally raised. This may reflect a condition functionally defined in terms of minimum shutdown time compared to maximum allowed pressure limit 152. However, pressure limit 153 is optionally fixed, e.g., to avoid possibility of doubt.

### Normal Operation

As shown in pressure state 160, and for purposes of discussion, the difference between pressure limits 153, 152 corresponds to twice the width of the arrow indicating the rate of gas inflow 141. In some embodiments, this corresponds to an assumed constraint that it takes “two arrow times” (e.g., 50-100 msec if gas inflow 141 represents pressure change per 25-50 msec) to shut down plasma gas delivery.

In some embodiment, adjusting the rate of gas inflow 141 is not available as a degree of freedom to the function of pressure regulation (optionally except to shut it down). Accordingly, the remaining state examples of FIG. 2A assume this is a fixed value, unless shutdown has been initiated.

In some embodiments, maintaining the balance of gas inflow 141 and gas outflow 142, as shown in state 160, comprises balancing the given (e.g., effectively constant) rate of gas inflow 141 by the adjustment of one or more mechanisms regulating the rate of gas outflow 142.

These mechanisms optionally comprise manual balancing; e.g., hand operation of a stopcock valve, for example as described in relation to FIG. 2B. However, based on manual balancing alone, there may be a tendency for pressure to continually rise or continually fall, unless the balance point is found exactly. This tendency is potentially mitigated if the system equilibrium points are inside the operating pressure range. For example, if pressure drops because gas is being exhausted too fast through a valve, the rate of gas exhaust may itself reduce, so that at some point, gas outflow 142 comes into equilibrium with gas inflow 141. Furthermore, e.g., once an equilibrium point is found, further adjustments of the stopcock valve may optionally be understood as adjusting not only the rate of gas outflow 142, but also the equilibrium pressure, other things remaining equal (for example, to reduce pressurization of tissue and/or change the insufflation state of the target treatment region. Examples of how this may be achieved in design are discussed, for example, in relation to FIG. 2B.

Additionally or alternatively, mechanisms for maintaining a target pressure optionally comprise automatically adjustable elements which are operated (e.g., based on pressure sensing) to maintain a pressure set point or target pressure range. For example, adjustment may comprise modifying the opening state of a valve, and/or other modification of a flow pathway, e.g., to increase or decrease flow resistance.

Whether manual or active, mechanisms including those just mentioned are usable with either or both of “passive” (against ambient pressure) and “active” (against vacuum pressure) evacuation control arrangements.

In some embodiments, moreover, pressure conditions (e.g., vacuum conditions) are actively generated using a pump and/or other means; these comprise another group of mechanisms which may be used to regulate pressure in some embodiments of the present disclosure.

### Potential Control Limitations

In the normal operation case of pressure state 160, there may be no demands on control (e.g., because an equilibrium has been set), or relatively low demands on control. For an automatically controlled system, “low demands” may mean that any environmental (e.g., pressure) changes which do require control intervention are slow and/or moderated enough that several response cycles of measurement (sensing), decision making (determining), adjustment (controlling), and system response (responding) are available. For example, 5, 10 or more cycles may be available to assess and react to a certain pressure change before a serious problem (e.g., potential exit from a targeted range of pressures before the next such cycle can complete) is in view. For a manually controlled system, “low demands” on control may mean that there is time to issue a user warning (e.g., a light, message, or buzzer) that the user reacts to only after a few seconds (e.g., 10 seconds or more). In some cases, plasma generation optionally occurs without inflow of ionization gas; for example, if the environment of target treatment region 112 is itself filled with a gas composition suitable for plasma ignition.

However, some potential scenarios represent exceptions outside of these parameters. It is an object of some embodiments of the present disclosure to decrease the time needed to handle them, and/or increase the certainty with which they are safely managed.

It is noted that manual adjustment is inherently slow to react to changing conditions, e.g., compared to a target response time in the sub-second (e.g., 100 msec or less, 10 msec or less) time regime. In any case, manual adjustment implies a demand on operator attention which it is a potential benefit to avoid. Manual adjustment, if relied on solely, potentially results in excessive shutdowns and/or equilibrium instability (e.g., system chatter and/or “rattling” of the treated area).

Moreover, even the response time of automatic adjustment is potentially limited, depending on the automatic mechanisms available. In defining this time (and the associate “response cycle”, which repeats steps of sensing, determining, controlling, and responding), there is, to begin with, a potential upper limit on the speed with which change is dependably measured. This can depend, for example, on the sensor itself, its location, and/or the slowest dependable rate at which it is polled.

Particularly with respect to safety design, the worst case limit on sensing and/or processing of sensed information may of critical concern. This can be affected by considerations such as the speed of control circuitry and/or programming operation; and/or delays under special circumstances such as the co-occurrence of events which competing for processing resources. For example, competing interrupt handing routines of control circuitry may increase the handling time of an exception under certain circumstances. Worst case conditions considered in safety design also potentially apply to the evaluation of human performance.

Next, the controlled hardware may have time-limited response characteristics. For example, a pump cycling at five times a second may not always be instantly available to increase its pumping if the requirement comes during a low part of its cycle.

Furthermore, there can be lags due to time needed to generate a certain pressure response inside the system, and particularly within target treatment region 112; e.g., lags affected by the ratio of conduit and other internal volume to the capacity of a pump or other pressure-developing subsystem. An organ enclosing the target treatment region may itself be clastic, such that some portion of a change in the amount of enclosed gas is absorbed by changes in volume.

Measuring (for example) may be continuously or near-continuously while other actions of the cycle are performed. There may be multiple new measurement time points available for use by the next response cycle. Furthermore, beginning the next response cycle does not necessarily wait for the full response to occur. However, this results in the potential for overcorrection as the results of previous adjustments finally accumulate. Application of cybernetic control theory is not necessarily able to prevent overshoot, at least insofar as either or both of (1) the parameters of a potentially adverse pressure event and (2) the total response characteristics of the system (which includes the target treatment region 112 itself) may be unknown, or at least not certainly known so as to sufficiently eliminate risk.

Additionally, controlled hardware may not always be precisely controlled and/or controllable. This may be particularly so when it is operated in short response cycles; and/or when balancing hardware performance against costs of setup, maintenance, and/or the hardware itself. Certain pressure-controlling actuators such as certain types of valves and/or pumps may be controllable only to a rough degree; for example only open or closed (for a valve), “more open” and/or “less open” (for a valve), and/or “faster” or “slower” (for a pump), without precise control over how this affects system pressure and/or evacuation rate. Even if precision is possible via cybernetic control over relatively long time frames (e.g., over a multiplicity of cycles of measurement and adjustment), the near-instantaneous (e.g., single-cycle) effects of device control are potentially unknown and/or incompletely calibrated.

Finally, the nature of disturbances being responded to are potentially unknown (e.g., random, rapid, and/or complex in frequency). For example, a measured excursion of pressure from a targeted range (and/or towards one of its limits) could be the beginning of a trend which becomes more extreme, or it could be transient and self-correcting. Moreover, a trend is not necessarily predictable from its history; and/or it may be necessary to respond to the trend on the assumption that it could develop to a worse case, which potentially leads to risks of overcorrection.

### Example of an Urgent Pressure Excursion

The remaining examples of FIG. 2A illustrate a pressure excursion potentially leading to device shutdown in a system having at least automatic sensing and control of a valve or other rapidly actuated flow control element. Evacuation is optionally passive or active. Manual control may be available, but is not considered to be exerted during the course of the changes from pressure states 160-164.

In state 161, there has not been any adjustment to gas inflow 141 or gas outflow 142, but pressure has suddenly risen (relative to state 160) to a concerning but not yet critical level (that is still within the upper limit of pressure limit 152A). This pressure event may be due, for example, to external pressure being placed on a bladder through pressure exerted on the body of the treatment subject.

It should be understood that a pressure event could, additionally or alternatively, be otherwise induced; for example, by a change in the balance of gas inflow 141 and gas outflow 142, whether intentional or inadvertent; measured or unmeasured. Optionally, the “worst case” which is to be handled envisions a sudden unanticipated blockage of gas outflow 142 (e.g., a due to a tube that is kinked, a sudden blockage by fluid or other material, and/or collapse under weight); such that gas outflow 142 suddenly falls to zero, or another much lower than normal level, e.g., 75%, 50%, or 25% of its steady-state level.

Optionally, the “worst case” envisions a concomitant event worse than 100% loss of outflow: for example 100% loss of outflow along with a decrease in the volume of the target treatment region (e.g., due to external pressure which squeezes it), such that up to a 110%, 120% or other “effective” loss in gas outflow 142 is to be handled. It is noted that “sudden” is optionally immediate (i.e., within a single response cycle) or rapid (e.g., within a number of response cycles, e.g., 5, 10, 20 or more). Optionally a more sudden case substitutes for consideration of a less sudden case with a potentially larger effect on outflow. The duration of the response cycle of concern is optionally selected according to the scenario of concern, actual regulating performance of plasma system 102 (optionally assumed degraded in a certain scenario), its ancillary components, and/or the target treatment region 112 to which it is coupled. For example, a response cycle time of about 1 msec, 5 msec, 10 msec, 20 msec, 50 msec, 100 msec, or more may be assumed.

Pressure state 162 represents a first option for responding to this situation, according to some embodiments of the present disclosure. It is assumed in this case that the pressurization event of state 161 has continued to increase, but only by a moderate amount. Still in this case, it has not become necessary to modify gas inflow 141; i.e., plasma delivery is continuing unmodified and uninterrupted. However, the rate of gas outflow 142 has been dramatically increased—e.g., automatically, and either actively or passively. This increase goes well beyond the level needed to restore an equilibrium. The parameters of the pressure increase may be unknown, and/or the amplitude of the response be under only rough control.

However, in some embodiments, the initial response has the potential advantage of being rapidly activated; e.g., responding within a response cycle (1 msec, 5 msec, 10 msec, 50 msec, or another time). For example, this may be the case for an electronically activated valve, which until now has been at least partially closed so as to restrict the rate of gas outflow 142.

By the time of pressure state 163, the pressure event has passed, and the previously opened valve or otherwise actuated device has been closed again, restoring the original control state of the hardware. Since pressure is reduced, so is the rate of gas outflow 142. When pressure state 164 is reached, elevating pressure has elevated gas outflow 142 to restore an equilibrium.

### Variations on Pressure Excursions

There are also certain alternative outcomes which could happen, in some embodiments; for example, “could” insofar as they are considered in the design the risk-mitigation performance of the system. A non-exhaustive list of examples is briefly provided next.

### Initial Overpressure and Response

### Regarding the Initial Pressure Event:

The pressure in state 161 could have been higher or lower before actions were initiated (but still not critical). Optionally, actions to increase gas outflow 142 could have been correspondingly more or less aggressive; e.g., opening a valve more or opening more valves (for passive regulation), and/or (actively) lowering the exhaust-side pressure, for example by increasing the output of a pump, or opening a valve leading to an existing low-pressure compartment.

The same actions would optionally apply if the pressure in state 161 was above pressure limit 152A (outside the targeted range) but not yet requiring shutdown. Additionally, in some embodiments, less care is exercised to ensure that there is no overshoot on the lower side of the target pressure limit 151A. For example, this amplitude of pressure excursion, should it occur, may be assumed to have a potentially more severe associated “worst case” of continuing trend, so that risking collapse and/or crossing a shutdown threshold of pressure limit 151 is preferable.

If the pressure at state 161 had reached or exceeded pressure limit 153, shutdown of ionization gas outflow 142 would have been triggered, e.g., on the design assumption that avoiding later crossing pressure limit 152 is no longer completely assured under worst considered case assumptions. Automatic recovery from this after initiation is not excluded if further monitoring warrants; but preferably this is considered a critical fault condition, requiring operator input before continuing.

If the pressure had reached or exceeded pressure limit 153, shutdown of ionization gas outflow 142 would have been triggered, on the design assumption that avoiding later crossing pressure limit 152 is no longer completely assured under worst considered case assumptions.

### Overpressure Recovery

Each of these potentially leads to a different balance of inflow and outflow at pressure state 162, leading to (some version of) pressure state 163 in a longer or shorter time. Corresponding to pressure state 163, again different scenarios are possibilities, separately or together as appropriate, for example:

Just as there might have been a larger or smaller adjustment to increase gas outflow 142 after state 161, the pressure response itself might have been larger or smaller than shown (regardless of the action actually taken). Potentially there is no undershoot of the pressure level 150 at pressure state 164 at all. Potentially there is a larger undershoot, but it is within acceptable limits.

In any of those cases, there may have been further modulations of gas outflow 142. For example, a slower-modulating adjustment such pump speed may have occurred. It could have been activated to augment the initial increase in gas outflow 142; and/or to counteract it, e.g., to reduce-the-increase, which potentially helps to reduce overshoot and/or speed restoration of equilibrium. In some embodiments, both the valve and pump receive commands simultaneously, but the pump (or other slow-acting adjustment) takes more time to reach full effect.

Possibly one or more of the actuated responses between pressure state 161 and pressure state 163 are reliant on a condition further to the current value of pressure level 150 before resetting. For example, a pop-valve might actuate automatically (potentially as a matter of its mechanical design, even without external control) on reaching an overpressure, but require manual resetting. Until then, this could result in a new equilibrium pressure; for example, flow through the pop valve might be calibrated to reduce pressure to a level nearer to pressure limit 151A. In some embodiments, restoration of “normal” resistance controlling gas outflow 142 can be automatically restored, but restoration is reliant on a further condition, e.g., user confirmation, the passage of time, the stabilization of pressure, or another criterion.

Finally, the correction made after pressure state 161 could have been so severe that the possibility of recovery was abandoned as a matter of control logic (e.g., flow 141 has been/is being shut down), or is unavailable because the system 102 has exceeded its capacity to recover (e.g., pressure level 150 in a variation of pressure state 163 reaches below pressure limit 151, optionally initiating shutdown and/or a recovery mode).

It is not excluded that the states of the system could oscillate above and below an equilibrium value (e.g., under the influence of cybernetic lag) before settling. In some embodiments, oscillation itself comprises a condition which is noted over the course of several measurements, and is optionally reacted to, e.g., by damping control responses to reduce the oscillation. Optionally, oscillation itself (e.g., oscillation of a sufficient amplitude) is treated as concerning enough to warrant a more serious response; for example, dropping target pressure to a lower end of the target range, optionally initiating a recovery mode, or initiating termination of ionization gas inflow (plasma delivery) itself.

### Passive Exhaust Control of Ionizing Gas Insufflation

### Passive Exhaust Control System Overview

Reference is now made to FIG. 2B, which schematically represents a system 100 for plasma delivery to a target treatment region 112 with manual (and optionally automatic) passive exhaust control using a tap 216, according to some embodiments of the present disclosure. Alternative automatic control is optionally implemented via control line 217.

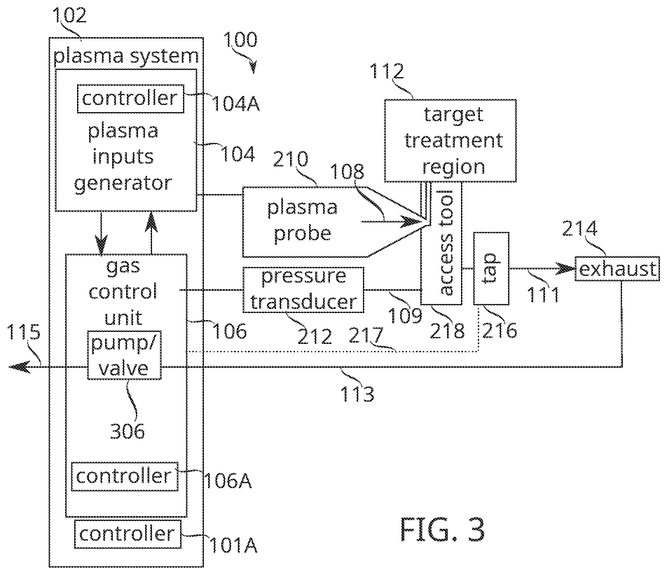

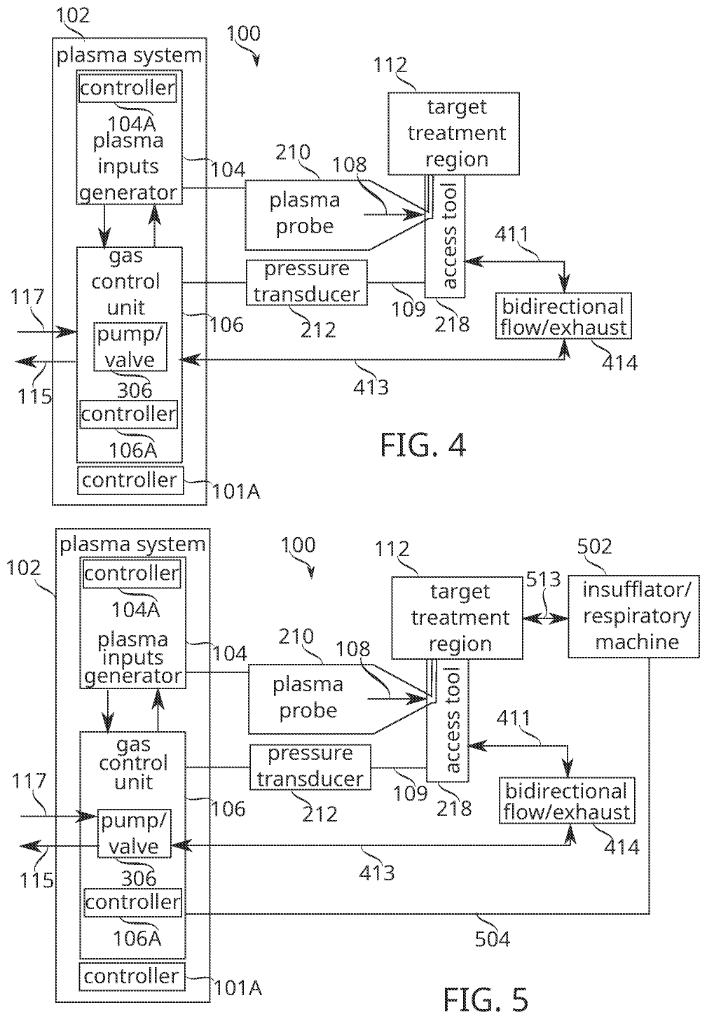

It should be understood that the discussion of passive exhaust control with respect to FIG. 2B is illustrative of certain principles and implementation options; and that the functions and features discussed in relation to FIG. 2B are optionally combined with (and not exclusive of) features described in relation to active evacuation; automatic and/or manually controlled. Insofar as components described in implementations are apparently only suitable for one or more of these modes of operation (but not all), the option for duplication of a function in an implemented form which is suitable should be considered available, except insofar as may be specifically stated and/or physically inconsistent. The converse of this understanding of the availability of mutual combination of embodiments also applies, changed as appropriate, for descriptions relating specifically to the active modes of exhaust control (e.g., but not exclusively, FIG. 3 and the bidirectional embodiments of FIGS. 4 and 5).

Reasons for combination include provision of a plurality of modes and/or mechanisms of gaseous environment control (e.g., pressure control) which complement each others' capabilities and tradeoffs. For example, modes of adjustment may be relatively slower or faster, relatively lesser or more precise in adjustment, relatively more or less reactive to the decision making of the operator, relatively more or less stable, and/or relatively more or less independent of a requirement for operator input. Examples described herein with respect to such capabilities and tradeoffs should be considered as non-limiting illustrations of the scope of combinations which are contemplated.

In the example of FIG. 2B, the plasma probe 210 which provides the pathway of the ionization gas flow 108 of FIG. 1 to the target treatment region is represent diagrammatically, including passage into/alongside an access tool 218 (e.g., a catheter, cannula, or other device) which provides access into target treatment region 112.

In the example of FIG. 2B, one direction of the management gas flow 110 of FIG. 1 is represented as a gas exhaust flow 111; passive in this case. Sensing data for use by plasma system 102 is provided via pressure access 109 of pressure transducer 212 to conditions in and/or in pressure communication with target treatment region 112. In some embodiments, pressure access 109 comprises a channel (e.g., with a diameter around 0.1-8.0 mm) which is inserted to target treatment region 112, optionally alongside conduits providing flow. A larger line diameter may help prevent inadvertent blockage conditions. This allows static pressure sensing, while a pressure transducer 212 placed at the flow lines themselves may require dynamic compensation and result in more indirect indications of pressure.

Ionization gas flow 108 to the target treatment region 112 through plasma probe 210 and/or access tool 218 is supplied from plasma inputs generator 104. Although access tool 218 is shown as a single block, this is not limiting; for example, there may be separate access tools for each of the ionization gas flow 108, pressure access 109, and gas exhaust flow 111. Optionally, two or more of the gas flows and/or sensing accesses share an access tool 218. Optionally one or more of these is divided among two or more access tools 218. In some embodiments, one or more of the access tools 218 comprises an endoscope. In some embodiments, an access tool 218 comprises a cannula, arthroscope, colonoscope, bronchoscope, catheter, or other device used to access internal regions of a living body.

Pressure transducer 212 indicates and/or estimates pressure at the target treatment region. Pressure transducer 212 may be positioned externally to access tool 218 and/or target treatment region 112 as shown; or in another position, e.g., mounted on plasma probe 210 and/or one of the access tools 218. In some embodiments, pressure transducer 212 is itself positioned at a distal tip of a plasma probe 210 and/or access tool 218, communicating, e.g., via wired connection (e.g., 0.1-8.0 mm diameter) back along one of these to interconnect with plasma system 102.

In some embodiments, a plurality of pressure transducers 212 are provided, decoupled in their sensing, for example, by placing them in different sensing locations, running their sensing lines along different routes (e.g., along different lumens of an endoscope, and/or monitoring them using different sensing circuits. In some embodiments, sensors based on different technologies are used; for example: pressure transducer, fiber optics pressure sensor, and/or on-tip micro-transducer. This potentially reduces the chances of a single fault failure encompassing all sensors.

One or more of the pressure transducers 212 is used as feedback inputs. When they are found to differ in readings by more than an expected amount, this is optionally considered as a fault, leading to entry into a recovery mode (e.g., reduced gas flow delivery and/or reduced pressure in target treatment region 112) and/or shutting down of the delivery of ionization gas flow 108.

Outflowing as exhaust flow 111 is potentially vulnerable to a failure mode in which fluid enters it from target treatment region 112, lowering its evacuation rate. In some embodiments, fluid ingress is mitigated by positioning a fluid evacuation lumen (not shown) with one or more inlets (apertures) located where they will collect fluid before enough builds up to be ingested by the main exhaust flow conduit(s). For example, one or more such apertures are positioned appropriate proximal (e.g., 5-50 mm) to main exhaust flow inlets (apertures) along a longitudinal axis of the flow conduits, so that they rest nearer to tissue surfaces that may collect fluids. The conduit which is adapted particularly to collect fluid is optionally operated at a sufficiently restricted flow rate so as to pick up fluids without making a major contribution to the overall volume of gas flow. Accordingly, should it (rather than the main gas conduit) become congested with fluid, the effect on the balance of flow is potentially mitigated.

Outflowing gas exhaust flow 111, in some embodiments, is controlled by the closing, opening and/or partial opening of tap 216. This may be performed manually; and/or automatically via control line 217. There is optionally more than one tap 216. The taps optionally have different capacities and/or controllability; for example: automatic/manual or both, on/off vs. continuous, relatively more or less precise, and/or relatively more or less rapid in operation.

Exhaust 214 indicates any suitable sink for the gas exhaust flow 111, e.g., the room or another open environment, a system of scrubbers/filters, and/or scavenging equipment to recover gas species as appropriate.

Optionally, system 100 of FIG. 2B is operated to deliver plasma as prescribed for a treatment procedure; e.g., for treatment of a tumor, infection, or other medical condition. The period of plasma delivery optionally comprises periods of constant, intermittent, and/or pulsing ionization gas delivery. Constant ionization gas delivery is preferred in some embodiments. Generation of plasma typically follows the envelope of ionization gas delivery, although within that envelope it is optionally intermittent (e.g. pulsed, or turned off for periods of seconds or more).

During the period of plasma delivery, the target treatment region 112 may be insufflated, e.g., to maintain visual and/or plasma probe access to regions of tissue within the target treatment region. It is anticipated that sufficient insufflation gas will be provided as a result of ionization gas supply from plasma inputs generator 104. However, auxiliary insufflation gas is optionally supplied from gas control unit 106 via management gas flow 110 and an optional additional conduit leading into target treatment region 112 (not shown).

The rate of outflow of gas exhaust flow 111 is managed, in some embodiments, by the setting of tap 216 to regulate the rate of passive outflow of gas exhaust flow 111. For example, tap 216 is implemented by one or more stopcocks, and/or other adjustable openings. In some embodiments, tap 216 is configured to release gas from a lumen of access tool 218. In some embodiments, tap 216 is configured to release gas from a dedicated line leading from target treatment region 112 itself.

The setting of tap 216 determines whether there is allowed minimal outflow, full outflow (that is, full-capacity outflow, according to the conditions of pressure and the flow pathways themselves), or intermediate outflow (i.e., outflow within a range between no outflow and full outflow).

In some embodiments of the present disclosure, tap 216 is constructed and/or configured so that its available range of adjustment corresponds to a safe range of equilibrated (steady-state) pressures inside target treatment region 112. Optionally, the most-closed state of tap 216 allowed is at least partially open, and set such that rate of gas outflow 142 (e.g., as described in relation to FIG. 2A) equilibrates with the rate of gas inflow 141 (e.g., ionization gas flow 108) at a pressure at or below pressure limit 152A. In some embodiments, the design of the most-closed state (e.g., the position of a motion stop and/or the capacity of a bypass element of tap 216) is determined by design. In some embodiments, additionally or alternatively, it is determined by calibration.

In particular, the adjustment range of tap 216 is optionally matched to an anticipated rate of gas inflow 141 and/or pressure. The adjustment range is optionally matched by a provided module embodying tap 216 which has the targeted properties inherently, and/or with a provided range of available calibration adjustments which are expected to ensure remaining at least reasonably close to the targeted working pressure even if uncalibrated (e.g., no more than 10%, 25%, or 50% beyond the upper end of the targeted working pressure, relative to ambient pressure. In some embodiments, tap 216 is configurable to match a particular working state of plasma system 102, e.g., according to the targeted rate of gas inflow 141, optionally as modified by considerations such as different flow resistances of access tool 218 as it may be modified or exchanged in use of plasma system 102 with different target treatment regions 112.

In some embodiments, tap 216 is optionally matched to a particular flow resistance of other system components such as the length and/or diameter of a lumen of access tool 218, and/or the length and/or diameter of conduits leading to lower pressure as set by ambient and/or active evacuation (e.g., as described in relation to FIG. 3). Optionally, tap 216 is provided with one or more flow restrictors (e.g., an auxiliary valve; and/or adjustable and/or selectable tubing of different lengths and/or diameters) which add different and/or variable amounts of resistance to gas outflow 142 in series with tap 216 that tunes the operating range of tap 216. Additionally or alternatively, the system is provided with one or more flow bypasses (e.g., a parallel path of gas flow, or an upstream or downstream exhaust location) which can be exchanged with each other, added, removed, and/or adjusted.

In some embodiments, tap 216 is configured to operate normally with a proximal side at below-atmospheric pressures. The matching of tap 216 to these conditions optionally takes this into account. In some embodiments (e.g., as a safety measure), a portion of the flow resistance and/or flow bypass associated with the configuration of tap 216 is constructed to open automatically upon full or partial loss of the expected below-atmospheric pressure. For example, a pop-valve may be held closed by vacuum against spring pressure tending to release it, such that if the vacuum is lost, the spring opens the valve, converting it to an exhaust vent.

In some embodiments, tap 216 is positioned at an outlet of a lumen of an access tool 218. Optionally, more than one outlet controlled by a plurality of taps 216 provided. Different outlets of tap 216 may each allow a different maximum amount of gas outflow, e.g., relatively coarse- and fine-adjustment outlets of tap 216, and/or relatively greater and smaller amounts of maximum exhaust flow passing through each outlet. Individual outlets of tap 216 may be configured so that they operate as substantially completely open or completely closed, with the rate of outflow being adjusted by which and/or how many outlets of tap 216 are open.

Optionally, tap 216 is at least in part manually set and/or manually adjustable. In one mode of operation, plasma system 102 is set to generate an approximately constant rate of supply of total gases (e.g., ionization gas flow 108) into target treatment region 112, such that manual adjustment of the setting of tap 216 can be used to modify the resultant pressure (and, e.g., corresponding state of insufflation). Accordingly a new state of equilibrium is potentially set. In some embodiments, automatic control of pressure is sufficiently relaxed so to allow such changes without seeking to restore the previous set point. For example, it detects that manual control was exerted according to an encoder, according to an indication of contact with the valve itself (e.g., a capacitance change), according to an input provided by the user such as a “hold” button, and/or according to a characteristic features of the adjustment itself (e.g., its time-course, for example, its speed and/or amplitude).

Optionally, the automatic control subsystem(s) of the plasma delivery system reacts in cases where pressure approaches a limit of the operating range of the device; restricting further adjustment, and/or moving the equilibrium pressure of treatment target region 112 further away from the limit approached.

Optionally, pressure measured by pressure transducer 212 is communicated to a user (e.g., displayed on a computerised display, and/or directly on by the transducer mechanism itself) for use in decision making about changes in manual settings. Additionally or alternatively, there is automatic control of tap 216 (exercised via control line 217, for example, in response to sensing by pressure transducer 212). In some embodiments, measurements from pressure transducer 212 are used as a basis for control of plasma inputs generator 104; for example, to initiate shutdown of ionization gas flow 108 in case a margin of environmental safety (e.g., guaranteed time to safely regulate pressure in target treatment region 112) is exceeded.

There may be unregulated exhaust paths as well, not shown explicitly, but corresponding to leakage 107 of FIG. 1. Optionally, flow through these is relatively restricted, so that the setting of tap 216 can be depended on to dominate control of gas exhaust.

### Active Exhaust Control of Ionizing Gas Insufflation

Reference is now made to FIG. 3, which schematically represents a system 100 for plasma delivery to a target treatment region with active evacuation control, according to some embodiments of the present disclosure.

The elements of the example of FIG. 3 are the same as described in relation to FIG. 2B, with modifications as next described.

In some embodiments, gas control unit 106 is further provided with its own active gas evacuation conduit 113, and evacuated gas exhaust 115, which also comprise part of the management gas flow 110 of FIG. 1. Active gas evacuation conduit 113 is configured to divert exhaust 214 to exhaust 115.

In some embodiments, negative pressure (that is, negative gauge pressure, compared to ambient pressure) applied to the proximal side of active gas evacuation conduit 113 is adjustable by the operation of a pump/valve arrangement 306 under the control of gas control unit 106. For example, the pressure is drawn down below atmospheric pressure, potentially increasing the maximum available throughput of tap 216, which remains controllable according to one or more of the methods and arrangements described in relation to FIG. 2B.

In some embodiments, pump/valve arrangement 306 comprises an active pump (e.g., a medical grade diaphragm pump). Additionally or alternatively, In some embodiments, pump/valve arrangement 306 comprises one or more valves which moderate pressure applied by the active pump, and/or by an external low pressure source such as a wall vacuum port of a clinical room.

In some embodiments, a pump of pump/valve arrangement 306 normally operates to pump a mixture of gasses comprising ionization gas flow 108 (which fluxes through the luminal space of target treatment region 112), and gasses from a second source; for example, ambient gas or a second gas supply. This provides the pump with basal operating conditions which keep it operating at a relatively large portion of its capacity in terms of movement of gas by mass compared to what is actually needed for evacuating luminal space 112. For example, under basal operating conditions, at least 10%, 25%, 50%, or another fraction of the pump's output comprises gas from the second source. For example, in the moments after a need for a rise in ionization gas evacuation capacity is detected, gas control unit 106 commands a valve of pump/valve arrangement 306 to switch to change the ratio of evacuation gas flow 108 to flow from the second source; increasing the contribution of the evacuation gas flow 108. Optionally, valve(s) are operated to restrict flow from the second source; optionally to increase flow from the evacuation gas flow; optionally both. The converse operations may occur to reduce evacuation of ionization gas flow 108.

In some embodiments, adjustment acts to maintain substantially the same overall conditions of pump operation (e.g., in terms of pressures generated and/or rate of gas exhausted), even though the mix of evacuated gases has changed. In any case (e.g., even if only one source is directly valved), the mix-in of gas from the second source during basal conditions also potentially serves to reduce the relative change in operating conditions. For example, a certain fractional change (e.g., increase) in the rate of evacuation of ionization gas flow 108 is optionally accompanied by a change in pump evacuation rate which is less than 50%, 25%, or 10% of that amount.

In the case of either increase or decrease: insofar as the pump is already operating at a capacity that moves a sufficient and suitable quantity of gas overall, it is not necessarily required to adjust its operation. Optionally, pump operation is also increased/decreased as appropriate. However, the time for this adjustment to make a meaningful difference in evacuation rate of ionization gas flow 108 is potentially subject to lag; for example, lag dependent on pump cycle time. As another potential benefit, the overall flow through pump is optionally set at or near optimal operating conditions for the pump, e.g., in terms of efficiency and or self-cooling.

Apart from response time of the hardware itself: by operating the pump above needed capacity, the system (e.g., in locations after the valves controlling relative flow) is placed already at a pressure low enough to meet a demand for increased evacuation of ionization gas flow 108, instead of having to develop over time to help meet the change in demand. As a result, lag is placed more directly under the control of valve actuation itself.

In some embodiments, vacuum is established by a device other than a system-dedicated pump; e.g. a building (wall) vacuum inlet, preferably buffered and/or suitably protected from contamination the system by a container acting as a trap and/or filter. Also in this case, it is a potential advantage to maintain basal conditions of flow which mix in gas from a second source, and to adjust ionization gas flow 108 by relative adjustment. For example, this potentially helps reduce proximal (outlet-side/vacuum-side) pressure variations, such as weakening after an initial increase in flow (e.g., when the valve to the ionization gas flow 108 is opened), or the reverse. This potentially assists in selecting stable and proportional responses to unintended changes in the pressure of treatment target region 112.

In some embodiments, there are provided one or more “reservoirs” of low pressure capacity established by pump/valve arrangement 306 or another device, which pump/valve arrangement 306 can expose to or block off from evacuation conduit 113 (e.g., by opening a valve), potentially providing a rapid-activation capability to moderate the rate of gas exhaust flow 111 (as an example of gas outflow 142). The valve optionally comprises, for example, a pinch valve, or another variable restriction mechanism, not necessarily with the capacity to entirely close off the exhaust conduit. In an other example, one or more valves may operate to switch in longer or shorter segments of evacuation conduit, and/or wider or narrower segments of evacuation conduit, in order to adjust exhaust flow rates.

This results in a configuration by means of which both tap 216 and pump/valve arrangement 306 exert a measure of control over the rate of evacuation of gas from target treatment region 112. Optionally, control line 217 is omitted, converting tap 216 to a purely manually operated device, with automated control exerted through the operation of pump/valve arrangement 306.

It is noted that as for tap 216, the capacity for flow restriction is optionally prevented from complete restriction of outflow. Optionally, it is calibrated to moderate flow within a regime which is reasonably expected to be consistent with (e.g., equal to) useful rates of ionization gas flow 108, while maintaining pressure in target treatment region 112 high enough, e.g., for insufflation, and in any case not drawing it down to a negative pressure; and low enough for safety. For example, pressures of 7-12 mmHg may be targeted. In some embodiments, pressures at least above 20 mmHg are to be avoided absolutely as a matter of safety and/or design. The absolute safety cutoff is optionally set lower than 20 mm (or whatever else the “maximum” safe pressure may be judged as).

Exhaust Control of Ionizing Gas Insufflation with Additional Inputs

### Additional Plasma-Generating Related Inputs

Reference is now made to FIG. 4, which schematically represents a system for plasma delivery to a target treatment region with bidirectional in-line gas control, according to some embodiments of the present disclosure.

The schematic of this embodiment includes the features of FIG. 3, modified to suppress tap 216, exhaust flow 111, evacuation conduit 113, exhaust 214 and control line 217. However, these should be considered to be provided (and subsumed in the embodiment of FIG. 4) in either or both of an active or passive configuration, and with any suitable combination of manual and automatic activation. More specifically, these elements are subsumed in the exhaust functions of bidirectional flow/exhaust 414, with further control and/or material introduction features, as next described. Features of monitoring and control (e.g., utilizing pressure transducer 212 and controllers of plasma system 102 and it components) are provided, e.g., as described in relation to FIGS. 1-3.

Flow control conduit 413 (e.g., subsuming gas evacuation conduit 113) comprises one or more conduits which form pathways for at least part of management gas flow 110, providing bidirectional flow/exhaust 414 (subsuming exhaust 214). Bidirectional gas flow 411 subsumes gas exhaust flow 111, connecting bidirectional flow/exhaust 414 to access tool(s) 218. In some embodiments, this arrangement is provided with all the evacuation capacity needed to manage evacuation of ionization gas flow 108; in some embodiments, it is auxiliary to other arrangements; e.g., those of FIG. 2B or FIG. 3. Bidirectional flow exhaust 414 is shown as managed by pump/valve arrangement 306, augmented as appropriate from the arrangements of FIG. 3 to perform the further functionality next described. For example, pumps are optionally operated bidirectionally, and/or positive pressure sources are optionally provided to drive material into target treatment region 112.

As described in other embodiments, plasma delivery is preferably performed according to its own prescribed delivery regime, with the flow of plasma gas being, e.g., constant, intermittent, and/or pulsating as appropriate. Preferably it is not modulated as part of pressure control, except in exceptional cases such as a requirement for device shutdown. However, it may be moderated according to plan in order to assist introduction of materials via bidirectional flow/exhaust 414.

Also as before, plasma delivery (via plasma probe 210) occurs together with insufflation and automatic target treatment region pressure and composition control. In some embodiments, one or more access tool(s) 218 provide endoscopic access, as well as sufficient further luminal capacity e.g., making use of splitter and/or further luminal channels and/or ports as appropriate) to evacuate the delivered ionization gas flow 108.

Bidirectional flow/exhaust 414 serves to introduce materials 117 to target treatment region 112 other than the plasma inputs which plasma inputs generator 104 supplies. This optionally comprises, for example, gas of a composition which mixes with the ionization gas from the plasma probe to generate suitable reactive species. For example, the supplied gas optionally comprises nitrogen and/or oxygen species which undergo conversion such as conversion to free radicals and/or ionization upon interacting with generated plasma. Optionally, the introduced materials perform humidification.

To provide these materials, bidirectional flow/exhaust 414 optionally includes a dedicated line (inflowing), operated continuously or intermittently as the plasma delivery protocol specifies. Additionally or alternatively, in some embodiments, an exhaust conduit is periodically diverted to operate in reverse. For example, there are optionally provided a plurality of (divided) exhaust lines, of which a portion (e.g., one) is occasionally co-opted for reversed operation to delivery materials. In such a case, operation of the remaining evacuation lines is optionally boosted as needed, for example by steepening the pressure gradient over which they are evacuating material, reducing their resistance to flow (e.g., opening one or more valves) or otherwise increasing flow through them. Additionally or alternatively, safe pressure conditions are maintained by alternating the operation of plasma probe 210 (and delivery of ionization gas flow) with operation of bidirectional flow/exhaust 414 to deliver material. In some embodiments, pressure inside target treatment region 112 is first deliberately reduced to nearer a bottom end of an acceptable pressure range (e.g., nearer to 7 mmHg than 12 mmHg), allowing a short period during which evacuation (e.g., via bidirectional flow/exhaust 414) can be stopped or reduced so that material can be introduced. To reduce dead volume, a switching valve may be provided (e.g., on or at a port of access tool(s) 218) as near to the target treatment region as practical.

### Further Plasma-Generating Related Inputs

Reference is now made to FIG. 5, which schematically represents a system 100 for plasma delivery to a target treatment region 112 with automated gas control in coordination with an external insufflator/respiratory machine 502, according to some embodiments of the present disclosure. It should be understood that embodiments of insufflator/respiratory machine 502 are normally configured as just one of an insufflator or respiratory machine.

The schematic of this embodiment includes the features of FIG. 4 as described, including cumulation with the features of FIGS. 1-3. However, bidirectional flow/exhaust 414 (and its supporting features) is optional; e.g., the additional features next described are optionally provided to embodiments corresponding to FIG. 2B and/or 3.

In some embodiments, insufflator/respiratory machine 502 is provided, which operates, via auxiliary flow 513, to manipulate pressure and/or volume in target treatment region 112. In some embodiments, plasma delivery (e.g. ionization gas composition, pressure, and rate of flow) is maintained substantially as dictated by treatment parameters, without adjustment to accommodate the moment-to-moment operations of the external insufflator or respiratory machine. However, plasma delivery may be paused periodically to allow navigation and/or repositioning of the probe; and/or to accommodate the operations other device (preferably on a schedule). Optionally, operation of the insufflator/respiratory machine 502 is adjusted to augment and/or avoid interference with the delivery of plasma treatment. Communication and/or control is established via connection 504, allowing plasma system 102 to adjust the operation of insufflator/respiratory machine 502, and/or adjust its own operation, as appropriate.

Embodied as a respiratory machine, for example, insufflator/respiratory machine 502 may be operating to provide lung respiratory support while plasma is being simultaneously delivered to the lungs. The rate of evacuation of plasma gas (e.g., by pump/valve arrangement 306) is optionally reduced while respiratory machine 502 is in an expiration phase of it cycle.

Embodied as an insufflator, for example, insufflator/respiratory machine 502 may be monitored and/or operated via connection 504 to provide insufflation auxiliary to the insufflation which the ionization gas flow 108 provides, as appropriate. For example, it may provide: additional peak volume, insufflation maintenance while ionization gas flow 108 is paused, and/or supplemental insufflation to maintain a more constant level of insufflation in case ionization gas flow 108 is pulsed or otherwise variable.

In some embodiments, control is targeted at maintaining a particular proportion of gases, e.g., of insufflation/respiratory gases compared to gases used in plasma generation. It is noted that in more open-orifice (freer flow) settings such as in lung, the target of control may be less directed toward pressure, and more directed toward ensuring that there is, e.g., sufficient oxygen rich gas to breathe, while also delivering an ionization gas flow, potentially at a rate of flow large enough to be competing.

In some embodiments, control of insufflator/respiratory machine 502 may be unavailable. Optionally, gas control unit 104 itself provides evacuation compensation as appropriate, e.g., based on sensing of pressure, and/or signal-communicated indications of the operational phase of insufflator/respiratory machine 502.

### Pressure Safety and Starting Pressure Features

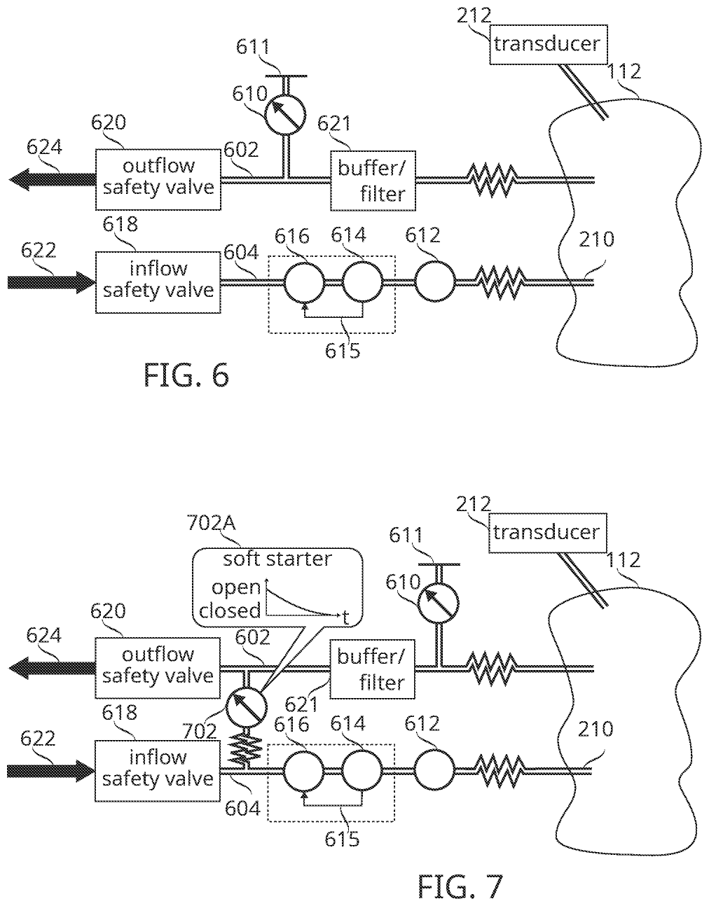

Reference is now made to FIG. 6, which schematically represents a system for plasma delivery to a target treatment region with automated evacuation control via inline vacuum, according to some embodiments of the present disclosure. Further reference is made to FIG. 7, which schematically represents a system for plasma delivery to a target treatment region with soft start cross-valve, according to some embodiments of the present disclosure.

In general, the embodiments of FIGS. 6 and 7 are optionally elaborations of the implementation of gas conduit interconnections between target treatment region 112 and a plasma system 102, specifically emphasizing safety and/or backup features, with inflow 622 to conduit 604 representing ionization gas flow 108 (with plasma probe 210 being at the distal end of conduit 604), and outflow 624 from conduit 602 representing the exhaust component of management gas flow 110. Transducer 212 is positioned and otherwise configured to measure pressure in target treatment region 112, and interconnected with suitable control circuitry (e.g., of a plasma system 102) which controls other mechanisms described with respect to other elements of this embodiment.

In some embodiments, one or both of inflow safety valve 618 and outflow safety valve 620 are provided. Activation to fully open outflow safety valve 620 can be used to quickly increase outflow in case a potential overpressure situation is detected. Conversely, it can optionally be closed, e.g., to prevent direct application of vacuum pressures to target treatment region 112. Activation is optionally under external control (e.g., in response to sensing by transducer 212), or under internal control (e.g., in response to a pressure differential sensed across the body of the valve).

In some embodiments, saturation valve 610 actuates to prevent and/or relieve out-of range pressure conditions. For example, it can open to vent overpressure from outflow conduit 602 to ambient pressure 611, optionally to compensate for closing of outflow safety valve 620, or another overpressure condition. Valve 610 may be opened, for example, in response to control exerted upon transducer 212 sensing a high pressure condition in target treatment region 112.

Buffer/filter 621 on outflow conduit 602 optionally serves to reduce contamination passing into the rest of the system, and/or prevent an excessively rapid development of negative pressure, should a flow imbalance favoring outflow 624 occur. In particular, this is of potential benefit during startup. With valve 610 open, vacuum pressure at outflow 624 can be activated; buffer 621 coupled and open valve 610 prevent its transmission to target treatment region 112. With an increase in inflow 622 and a suitable increase in pressure, e.g., sensed by transducer 212, valve 610 can be closed again, and the system flows allowed to equilibrate. Furthermore, valve 610 is optionally opened in case of an underpressure fault; e.g., to prevent vacuum pressure from developing on the distal side (right) of buffer/filter 621.

The arrangements of FIGS. 6 and 7 also include safety flow transducer 612, which provides a backup indication that the device is operating at least roughly within intended parameters. Upon receiving an unexpected indication from flow transducer 612 (e.g., excessive flow, reduced flow, or lack of flow when flow is expected), a system controller can ensure that the other valves of the system are set to a state which protects target treatment region 112. Additionally or alternatively, flow transducer 614 is configured to provide feedback control to the state of flow valve 616, e.g., at least to prevent exceeding and/or falling beneath a certain (consistent with safety) range of flow rates, and optionally to target a specific flow rate.

In some embodiments, an arrangement (FIG. 7) comprising valve 702 and an interconnection between outflow conduit 602 and inflow conduit 604 is provided, assisting in soft-start of inflow and outflow. Initially (e.g., upon insufflation start), valve 702 is open, allowing a significant portion of inflow to bypass target treatment region 112. This situation without active evacuation can persist for a long time, since it bypasses much of the evacuation resistance of conduit 602, and prevents over-pressurizing target treatment region 112. Vacuum pressures can then be applied to outflow conduit 602, similarly without transmitting vacuum pressure into target treatment region 112. Once the two flows are stabilized with respect to each other, valve 702 can be closed; i.e., gradually closed, for example while monitoring pressures/flows and adjusting to compensate. Buffering to increase startup time is potentially useful, e.g., for allowing a luminal space time to equilibrate (e.g., expand safely).

The control sequences described in relation to the mechanisms of FIG. 6-7 are preferably configured to operate under full automation.

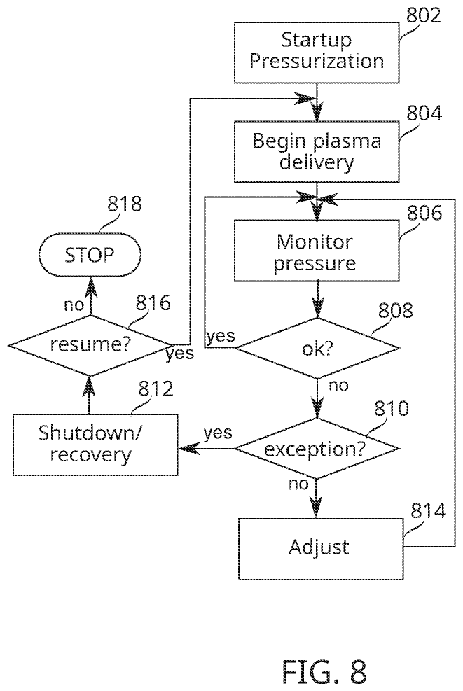

Reference is now made to FIG. 8, which schematically represents a flowchart for plasma delivery to a target treatment region, according to some embodiments of the present disclosure.

At block 802, in some embodiments, the flowchart begins. It is assumed that the system has generally been configured for operation already, optionally including positioning of both the plasma probe and the exhaust conduit where it and a startup pressurization routine is performed; for example startup pressurization as described with respect to FIGS. 6-7. Startup pressurization brings the flux of gases up to its operating level, while taking care to avoid imbalances of inflows and outflows as each reaches its initially targeted level.

At block 804, in some embodiments, plasma delivery begins. At this point, ionization gas flow 108 is at the predetermined level associated with the planned treatment protocol, and evacuation through gas exhaust flow 111 is in equilibrium with it (along with any uncontrolled leakage/evacuation which may be occurring).

At block 806, in some embodiments, pressure is monitored.

At block 808, in some embodiments, a decision is made if the pressure received is “ok”—that is, both within a nominal operating range, and not in need of adjustment. If it is ok, the flowchart returns to monitoring pressure at block 806.

Otherwise, in some embodiments, at block 810, a determination is made if the system is in a pressure exception system—that is a state in which safety considerations dictate switching to shutdown/recovery mode. If so, the system goes to block 812. Upon resumption (if elected and/or automatically occurring at block 816), the flowchart continues with block 804. Otherwise, the flowchart stops at block 818.

If the system does not enter an exception state, then at block 814, in some embodiments, an appropriate correction to the system state is made to bring the system back toward a target pressure and/or pressure range.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application was specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

What is claimed is:

1. A system for controlling luminal pressure during plasma dosage to a luminal space within tissue, the system comprising:
a plasma inputs generator, configured to provide to a plasma probe an ionization gas flow according to one or more parameters, the ionization gas flow sufficient to maintain the luminal space in an insufflated state;
the plasma probe, sized for insertion to the luminal space, and configured to receive the ionization gas flow, along with an electrical signal an electrical signal delivering power for plasma generation, to generate plasma within the luminal space;
at least one evacuation conduit, sized for insertion to the luminal space;
a pressure transducer, configured to indicate gaseous pressure in the luminal space; and
a gas control unit, coupled to the at least one evacuation conduit, and configured to maintain the indicated gaseous pressure in the luminal space within a targeted range of pressures by adjusting a rate of gas outflow from the luminal space through the at least one evacuation conduit, while plasma inputs generator continues to produce ionization gas flow according to the parameters.

2. The system of claim 1, wherein:
the gas control unit comprises a pump and at least one valve;
the pump operates to evacuate a gasses from the ionization gas flow mixed with gases from a second source bypassing the luminal space; and
the gas control unit adjusts the rate of gas outflow from the luminal space by actuating the valve to adjust a relative amount of gasses from the ionization gas flow and from the second source.

3. The system of claim 1, wherein the gas control unit:
operates to establish a low-pressure region with an exhaust pressure below atmospheric pressure, in non-equilibrium pressure communication with:
the luminal space, and
a second source of gas, bypassing the luminal space and at a pressure above the exhaust pressure;
comprises at least one valve regulating flow into the low-pressure region from at least one of the luminal space and the second source of gas; and
adjusts the rate of gas outflow by operating the at least one valve.

4. The system of claim 3, wherein, in a basal operating condition, flow from the second source of gas is at least 50% of the ionization gas flow passing into the low-pressure region with the exhaust pressure.

5. The system of claim 3, wherein the rate of exhaust of gasses through gas control unit from the low-pressure region with the exhaust pressure changes by less than 50% of corresponding changes in rate of gas flow gas outflow resulting from adjustment by gas control unit.

6. The system of claim 1, wherein the gas control unit maintains the targeted range of pressures by operation of a first pressure-control actuator and a second pressure-control actuator; wherein the first pressure-control actuator responds more rapidly than the second pressure-control actuator, and adjustment to the second pressure-control actuator has more controlled effects on pressure than adjustment of the first pressure-control actuator.

7. The system of claim 6, wherein at least one of the first and second pressure-control actuators is a valve, and at least one of the first and second pressure-control actuators is a pump.

8. The system of claim 1, comprising a manual valve adjustable to modify gas outflow and change the gaseous pressure in the luminal space to a new pressure within the targeted range of pressures; wherein adjustment performed by the gas control unit corrects to allow the new pressure to remain.

9. The system of claim 8, wherein, upon receiving an indication of a change from the new pressure toward a pressure beyond a threshold of the targeted range of pressures, the gas control unit adjusts the indicated gaseous pressure in the luminal space toward a pressure further from the threshold than the new pressure.

10. The system of claim 8, wherein a range of available adjustment of the manual valve is limited to prevent a manual change to a pressure outside the target range.

11. The system of claim 1, wherein the at least one evacuation conduit comprises at least first and second conduits; wherein inlets to the first and second conduits are separated along their axis of elongation so that inlets of the second conduit are positioned to collect fluid before the fluid reaches inlets of the first conduit; and where the first conduit carries the majority of exhaust flux of the ionization gas flow from the luminal space.

12. The system of claim 1, wherein gas control unit is configured to adjust the rate of gas outflow, based additionally on one or more sensed indications of the operation of at least one of an insufflator or respiratory machine.

13. The system of claim 1, comprising a conduit configured for delivery of material into the luminal space without going through the plasma probe and under control of the gas control unit; wherein the gas control unit adjusts the rate of gas flow to compensate for the delivered material.

14. A method of controlling luminal pressure during plasma dosage to a luminal space within tissue, the system comprising:
inserting the plasma probe into the luminal space;
operating a plasma inputs generator to provide to the plasma probe an ionization gas flow according to one or more parameters, the ionization gas flow sufficient to maintain the luminal space in an insufflated state, while the plasma probe generates plasma using the ionization gas flow and an electrical signal delivering power for plasma generation; and
while the plasma inputs generator continues to produce ionization gas flow according to the parameters, evacuating the ionization gas from the luminal space through at least one evacuation conduit under the control of a gas control unit;
wherein the evacuating comprises repeatedly:
receiving an indication of gaseous pressure in the luminal space; and
adjusting a rate of the evacuating, based on the received indication, to maintain the indicated gaseous pressure in the luminal space within a targeted range of pressures, allowing the plasma inputs generator to continue providing the ionization gas flow according to the one or more parameters.

15. The method of claim 14, wherein the gas control unit comprises a pump and at least one valve; and
the evacuating comprises evacuating a mix of gasses from the ionization gas flow and from a second source; and
the adjusting comprises actuating the valve to adjust a relative amount of gasses from the ionization gas flow and from the second source.

16. The method of claim 14, wherein:
the evacuating comprises establishing a low-pressure region under the control of the gas control unit with an exhaust pressure below atmospheric pressure, and in non-equilibrium pressure communication with:
the luminal space, and
a second source of gas at a pressure above the exhaust pressure; and
the adjusting comprises adjusting least one valve regulating flow from at least one of the luminal space and the second source of gas into the low-pressure region.

17. The method of claim 14, wherein the adjusting comprises actuating both a first pressure-control actuator and a second pressure control-actuator; wherein the first pressure-control actuator responds more rapidly than the second pressure-control actuator, and wherein adjusting the second pressure-control actuator has more controlled effects on pressure than adjusting of the first pressure-control actuator.

18. The method of claim 17, wherein actuating the first and second pressure-control actuators comprises adjusting both a pump and a valve.

19. The method of claim 14, wherein the evacuating comprises:
receiving an indication that a valve has been manually adjusted to modify gas outflow and change the gaseous pressure in the luminal space to a new pressure within the targeted range of pressures;
allowing the change in the gaseous pressure to remain as a new equilibrium pressure;
receiving an indication of a further change from the new pressure toward a pressure beyond a threshold of the targeted range of pressures; and
adjusting the indicated gaseous pressure in the luminal space toward a pressure further from the threshold than the new pressure.

20. The method of claim 14, wherein the at least one evacuation conduit comprises at least first and second conduits; and comprising:
positioning inlets to the first and second conduits with separation along their axis of elongation, so that inlets of the second conduit are positioned to collect fluid before the fluid reaches inlets of the first conduit.

21. The method of claim 14, wherein the adjusting comprises:
receiving an indication of the operation of at least one of an insufflator or respiratory machine to modify the gaseous pressure; and
adjusting the rate of gas outflow based also on the indication.
